(19) United States
(12) Patent Application Publication
(10) Pub. No.: US 2015/0342511 A1
(43) Pub. Date: Dec. 3, 2015

(54) OPERATING SYSTEM WITH COLOR-BASED HEALTH STATE THEMES

(71) Applicant: Neumitra Inc., Boston, MA (US)

(72) Inventor: Robert F. Goldberg, Boston, MA (US)

(73) Assignee: Neumitra Inc., Boston, MA (US)

(21) Appl. No.: 14/721,851

(22) Filed: May 26, 2015

Related U.S. Application Data
(60) Provisional application No. 62/002,758, filed on May 23, 2014.

(51) Int. Cl. A61B 5/16; G09B 19/00

(52) U.S. Cl. CPC A61B 5/165; G09B 19/00

(57) ABSTRACT

Techniques for implementing a platform configured to interact with a plurality of applications on a computing device to provide health information of an individual. The platform may be implemented by receiving information representative of a health state of an individual. The information may be provided by a sensor associated with the individual and provide data indicating the individual's physiological and/or emotional state. An indication of the individual's health state may be generated by selecting a value from a set of values representative of different health states. The indication may be provided through a programming interface to one or more applications. An application may provide context information associated with the individual at a time associated with the individual's health state, and the context information may be used to generate the indication.

### RELATED APPLICATIONS

This application claims priority under 35 U.S.C. §119(e) to U.S. Provisional Application Ser. No. 62/002,758, entitled “OPERATING SYSTEM WITH COLOR-BASED HEATH STATE THEMES” filed on May 23, 2014, which is herein incorporated by reference in its entirety.

### BACKGROUND

Biosensors measure physiological signals representative of a person's physiological and emotional health. This information may be used as a type of biofeedback, which may aid a person to be aware of and alter their response to stressful situations or to avoid those situations. This information may also be used for diagnosis, detection, monitoring or treatment of physiological disorders.

Biosensors may measure physiological signals such as temperature, pulse rate or sweat production of a user. The biosensors may be worn by a user such that they can measure those signals over time as the user participates in various activities. Such measurements produce data that may be analyzed to determine a user's biological and/or health state, such as if the user has a higher than average temperature.

### SUMMARY

Aspects of the present application are directed to a platform configured to perform a method for interacting with a plurality of applications on at least one computing device. For example, methods may allow for receiving information representative of a health state of an individual, generating an indication of the health state, and providing the indication to one or more applications.

According to an aspect of the present application, at least one non-transitory computer-readable storage medium storing computer-executable instructions that, when executed, implement a platform for at least one computing device is provided. The platform is configured to perform a method for interacting with a plurality of applications on the at least one computing device. The method comprises receiving information representative of a health state of an individual, receiving context information from at least one application of the plurality of applications, generating an indication of the health state, based, at least in part, on from the received health information and the context information by selecting a value from a set of values representative of different health states, and providing, through a programming interface, the indication to at least a portion of the plurality of applications.

According to an aspect of the present application, a portable electronic system associated with a user, the portable electronic system being configured to execute a plurality of applications is provided. The portable electronic system comprises at least one sensor configured to provide health information representative of a health state of the user. The portable electronic system further comprises at least one processor configured to receive context information from at least one application of the plurality of applications, generate an indication of the health state based, at least in part, on the health information and the context information, and present a platform interface common to the plurality of applications, wherein the platform interface is configured to enable the plurality of applications to access the indication.

According to an aspect of the present application a server comprising at least one processor configured to receive health information indicative of health states of at least one individual over time and at least one storage medium. The at least one storage medium stores processor-executable instructions that, when executed by the at least one processor, perform a method comprising identifying a portion of the health information based, at least in part, on user health data provided by at least one sensor associated with a user, generating an indicator for the user health data by analyzing the portion of health information and relating the user health data to the portion of health information, and transmitting the indicator to at least one portable electronic device over a wide area network, wherein the at least one portable electronic device is associated with the user.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic illustration of system including a platform that translates information indicating a health state of an individual into a representation of color;

FIG. 2 is a block diagram of components of a system interacting with a platform, including a biosensor that generates health state information and applications that receive from the platform and respond to a representation of color indicating the health state information;

FIG. 3 is a flowchart of an exemplary process of generating a color value used in a graphical user interface to represent health state based on a physiological signal according to some embodiments of the invention;

FIG. 4 is a flowchart of an alternative process of generating a color value used in a graphical user interface to represent health state based on a physiological signal;

FIG. 5 is a flowchart of an alternative process of generating a color value used in a graphical user interface to represent health state based on a physiological signal;

FIG. 6 is an illustrative example of the graphical user interface of an electronic device displaying a home screen with a daily overview having a color scheme corresponding to the user's health state for tasks associated with different applications;

FIG. 7 is an illustrative example of the user interface of an electronic device displaying a calendar application in month view showing a color scheme where each day has a color corresponding to a measured or expected health state of the user for that day;

FIG. 8 is an illustrative example of the user interface of an electronic device displaying a calendar application in day view showing a color scheme where sections of the day display a color corresponding to a measured or expected health state;

FIG. 9 is an illustrative example of a user interface displaying the health state of a user during the course of an activity in which the health state is indicated by a common color scheme;

FIG. 10 is an illustrative example of a user interface displaying applications where each application has a color representing a historical health state of the user when using the application;

FIG. 11 is an illustrative example of a user interface displaying a song list with a corresponding color for each song to indicate a user's historical health state associated with each song;

FIG. 12 is an illustrative example of a user interface displaying a map with colors corresponding to the health state of a user at each of multiple locations;

FIG. 13 is an illustrative example of an events overview, where each block corresponds to a type of event or activity and the color of a block indicates the health state of a user associated with that type of activity;

FIG. 14 is an illustrative example of a list of the instances of an event type where the color indicates the health state of a user for each event instance;

FIG. 15 is a functional block diagram of a computing system in which an operating system and applications as described herein may execute;

FIG. 16 is a flowchart of an exemplary process of generating a color value used in a graphical user interface to represent health state based on a physiological signal and population health data according to some embodiments;

FIG. 17 is a flowchart of an exemplary process of generating a color value used in a graphical user interface to represent health state based on a physiological signal and population health data selected by context information; and

FIG. 18 is a flowchart of an alternative process of generating a color value used in a graphical user interface to represent health state based on a physiological signal and historical health data selected by context information.

### DETAILED DESCRIPTION

The inventors have recognized and appreciated that the utility of a computing environment may be increased by providing health state information, including information indicating an emotional state of a person, in a way that enables unifying display and control mechanisms across multiple computer applications that humans interact with. Such an approach may improve the utility of a computing environment, both by providing additional information to a user and by enabling computer applications to readily adapt based on a user's health state. Accordingly, the inventors have developed a platform, which may be incorporated into a computer operating system, which receives information representative of a health state of an individual and generates an indication of that health state. The platform may execute on a portable electronic device that is associated with a user and may provide to computer applications health state information about the user, indicative, for example, of the user's emotional or physiological state.

The provided health state information may be represented in a way that enables the applications to provide a unified operating environment for the user in response to the health state information. In some embodiments, the operating environment may be unified by presenting interfaces for each of multiple applications that vary in a visual characteristic based on health state of a user interacting with that operating environment. In some embodiments, the health state information may be provided as a value from an enumerable set of values, and the applications may present information on a display in accordance with a visual characteristic corresponding to the provided value, or respond in any other suitable way, based on the provided value.

For example, the health state may be a mental state, such as an emotional state of a user. The platform may generate a value by selecting the value from a set of colors, with each color associated with an emotional state. As a specific example, the platform may be configured to detect, based on biosensor output and/or context information, a stress level of a user. The detected stress level may be mapped to a color, which may be provided to an application as a way to represent a health state of the user. As a specific example, the platform may output a value indicative of red when the health information indicates that the user is under stress. The hue of red may indicate a degree of stress. Conversely, the platform may output a value indicative of blue when the health information indicates that the user is relaxed. The hue of blue may indicate a degree to which the user is relaxed.

In some embodiments, a user's health information may be compared relative to a collection of previously acquired health information. The collection of health information may represent health data acquired from a population of multiple people and/or the user. The collected health information may be used to provide statistics when representing a user's health state. Such statistics may be used to determine a color value for the user's health state. The health states that a set of colors represent may be determined from health statistics computed from health data of a population. A user's health state may fall within the range of colors representing the population's variation in health, indicating the user's health relative to the variation in the population. For example, the population's health data may range over a set of health state values and this range of health state values may be used to determine the health state values represented by a set of color values. The hue of red may indicate one extreme of the range of health state values and the hue of blue may indicate the other extreme in the range. In this way, the color scheme may be normalized to the range of health state values of the population. A user's health state may be represented as a color value within the range of these extremes of the range of the health state values for the population. Additionally or alternatively, a user's health state may be normalized to the range of the population's health states and a normalized health state may be computed for the user in order to determine a representative color value. Each of the applications may use the provided health state information to tailor a display in accordance with the health state information. The operations and/or display may reflect a current, future, or prior health state of a user. For example, an application may generate a user interface using a color or colors indicative of a current health state of a user. Alternatively, the application may display a user interface with colors representing health state of the user at multiple prior points in time. As yet a further example, an application may perform operations associated with activities engaged in by the user. The application may generate and/or collect context information relating operation of the application at a time associated with the health information. One or more applications may provide context information that indicates the user's participation in a type of activity by the user's operation of the one or more applications. Context information obtained by applications may include information that the user is engaged in a particular type of activity, such as listening to music, playing a game, participating in a call or meeting, at a geographical location including traveling such as commuting to work. That application may associate health states with specific operations such that the application can determine that the user may enter the associated health state when those operations are invoked. By projecting when a user may perform an activity that will invoke that operation, or when that operation is scheduled to be invoked or is likely to be invoked, the application may predict a future health state of the user. Alternatively or additionally, if an application collects or generates context information such that it can identify that the user is performing the activity or directly predict when the user will perform the activity, the future health state might likewise be predicted. The predictions of future health state may be displayed, including using the same interface formats as with current health state information, but with a different timescale.

The application may alternatively or additionally use health state information, whether past, present, or future, to control in some way execution of operations by the application. For example, an application that plays music for a user may respond to health state information indicating a degree of stress above some threshold by selecting music that has historically, when played for the user, reduced the user's stress level. In this example, current user health state information is used to drive a selection of music to be played. Other applications may use health state information to select other actions, such as scheduling meetings or free time, blocking phone calls from being passed to a user or otherwise controlling execution of a task that may impact the user.

Similar control may be performed in response to predicted health state information. For predicted information, the control may occur in advance to prevent a user from entering an undesired health state or may be scheduled for when the user is predicted to be in a specific health state. In addition to controlling applications to perform operations with the potential to modify a health state, control based on health state information may be used to enhance a user's performance of certain activities. For example, by projecting that a user may be stressed at a particular time or otherwise not in a health state desired for engaging in certain activities, those activities may be scheduled by an application for another time. Alternatively, an application may schedule complementary activities such that periods of high stress and low stress are balanced. Such scheduling may be performed fully automatically or may be partially automated, such as by suggesting a schedule or rescheduling of activities to the user.

Further, the current health state information may be recorded to create a historical record of response to actions by the application, such as playing a particular song. This historical record may be accessed at a later time to predict impacts on a user's stress level associated with various actions that the application can control. Such automated addition of a user response to an action by an application may enable the application to select actions to modulate a user's health state. For example, when current health state information indicates that a user has a stress level above a threshold, an application may select an action to reduce stress level. Conversely, when current health state information is below a threshold, an application may select an action to raise stress level.

Automated control of applications based on health state information may rely in part on physiological information measured with biosensors. In some embodiments, the physiological information may be combined with context information. Context information may be derived from any suitable sources. In embodiments in which the physiological information is processed in a platform executing on a portable computing device that also executes applications, context information may be derived from the applications.

For example, a calendar application may provide information about planned activities of the user. A calendar application may indicate that a user is in different contexts, such as commuting or making an important presentation. The same physiological information may correlate with different health states in these different contexts. For example, an elevated pulse rate or sweat response in a commuting context may be undesirable and be reflected by the platform as a relatively high stress health state. The same pulse rate or sweat response in another context may indicate that the user is mentally alert and prepared for the important presentation.

Thus, in some embodiments, context information may be combined with physiological information to control action by applications. This information may be combined in the platform, such as by generating a relative health state output, indicating current health state relative to a target health state appropriate for a detected context. However, it should be appreciated that processing at its described herein may be performed in any suitable component. As an example of alternative processing architectures, the platform may provide to applications, along with health state information, context information. In this scenario, the application may determine the target health state based on context. In other scenarios, applications may generate context information rather than obtain it from the platform.

In some embodiments, processing of health state information may be personal to a single user. However, use of health state information is not limited in this way. In some embodiments, health state information may be aggregated for multiple users. Such aggregated health state information may be used when representing the user's health state. The aggregation may be based on health state information presented as one of a set of enumerated values. Such an approach may allow comparisons and aggregation of information about a large population. For example, a server managed by an enterprise may receive health state information, in combination with context or other information, about the population of individuals associated with that enterprise. Those individuals may be workers or customers of the enterprise, for example. Aggregated information may be presented graphically, such as by showing the aggregate health state for the population. Any suitable technique may be used to depict an aggregate health state. For example, an individual in the population may be represented as a point on a one, two or three dimensional plot with a color representative of the health state of the individual. The plot may be a geographic map, and may reveal locations at which individuals, in the aggregate, are stressed, excited or relaxed. Alternatively, the plot may correspond to a time scale and may reveal times at which the population the aggregate is in specific health states. Should be appreciated, however, that depicting individuals in the population as points on the plot is only one example of a suitable technique for displaying health state nation about a population in the aggregate. Other approaches include computing an average value for the health state of the individuals in the population and select being a color corresponding to the average value.

When health state information is aggregated for a population, context information for each health state value also may be aggregated. Such context information may be used to identify the context of the individual when particular health data is acquired. For example, such context information may include the individual's activity, geographical location, individual's age and/or occupation. In some instances, such context information may be used to select a subset of the health state values to provide statistics when representing a user's health state. The subset of health values may be selected such that the health data was acquired under similar contexts and have similar associated context information. Selecting a subset of health values by the context in which the health information was acquired may provide health statistics for the user under a similar context. The user's health state may be represented in a color scheme that reflects statistics computed from the subset of health values. A user's health state may then be represented by a hue in the color scheme to indicate the user's health relative to the health values in the subset.

A user's context information may be used to select the subset such that the context information of the health values in the subset has similar associated context information. The user's context information may be used to retrieve health data with associated context information similar to the user. For example, a population may include individuals of varying ages but a user may only want to select a subset of health data representative of other users of the user's own age. Other context information, such as location or activity in which the user is engaged, may be used to select the subset.

Alternatively or additionally, a user profile may be used to select the subset. A user may specify, such as by creating and storing a profile, which type of context information is used to determine a subset of health information. In such instances, there may be several user options indicating different context information that a user may select and/or rank in importance.

In some embodiments, the subset may be selected from health information aggregated for multiple people. The health information may be accumulated over time and/or may represent current health data of a population. A subset of the aggregated population health information may be selected to determine a color scheme for representing a current health state of a user. In such a color scheme, the user's current health state may be represented in comparison to the subset of the population health information.

In other embodiments, health information may be aggregated for an individual user over time to collect historical health information of the user. A subset of the historical health information of the user may be selected to determine a color scheme for the representing a current health state of the user. Using such a color scheme, a current health state may be represented in comparison to the previous health of the user.

The inventors have recognized that portable electronic devices, such as smartphones and tablets, have become a significant part of many people's daily lives. Applications executing of these devices may perform one or more tasks based on health state information. Moreover, an application executing on such a portable electronic device associated with the user may control tasks based on health state information. Because of the close association between the portable electronic device and the user, controlling execution of tasks on the portable electronic device may directly impact the user. Accordingly, embodiments as described herein may be implemented with a platform, such as a component of an operating system, executing on a portable electronic device.

In addition to other characteristics useful in generating or using health state information, portable electronic devices may contain sensors or other input mechanisms and execute numerous applications, any of which may generate information that may be used in determining health state of a user. For example, a speech processing application may, by analyzing cadence or other aspects of speech, provide information indicative of an emotional state of a user. As another example, an application that accesses an accelerometer, GPS or other sensor built into a portable electronic device may provide context information relating to the location or activities gauged by a user.

Moreover, applications executing on a portable electronic device may generate context information that may reveal health state information or may be used in applying health state information to control execution of tasks. Such portable electronic devices contain user information inputted by the user. For example, people use portable electronic devices to record and/or track many aspects of their daily activities. As a specific example, individuals may use a calendar application on a portable electronic device to keep track of their schedules, including past and future events. Calendar information may provide context information indicating what a user is doing or plans to do at any specific time. Many portable electronic devices also contain applications used by a user for particular activities, such as listening to music, calling another person, shopping, or running. Thus, these applications may provide context information useful in assessing, or controlling actions based on, health state.

Moreover, by providing health information to those applications, the applications may apply the health information in the context of activities that they control. Moreover, integrating information from physiological measurements with the user information in an application on a portable electronic device may enable a user to view their health status within the context of an application. The user may then be able to more easily and quickly see trends between their health and their behaviors and/or activities. When using a color scheme determined from population health information and/or a user's historical health information, a user may then be able to see how their health compares to the population's health and/or their previous health.

Likewise, the user and/or automated processing may correlate activities with health states such that a user and/or automated processing within the application may predict future health states based on activities that one or more applications indicate are planned to occur or are likely to occur. With this capability, a user and/or automated processing may control one or more actions, such as adjusting the schedule of upcoming activities to achieve a desired health state for the user. For example, by correlating meetings at a particular location or with a particular individual with health state, a calendar application may identify upcoming periods of time when a user may be under stress. This information may be used to adjust the user's schedule, such as by scheduling less stressful activities before or after those projected periods of stress.

To facilitate such control, the inventors have developed techniques that enable an electronic device to represent a health state in a common way among applications on the electronic device. By having a common representation, health information may be seamlessly integrated into the application information for each application. In some embodiments, the common representation may be a color code.

Health state information may be obtained in any suitable way. The health state information, for example, may be determined from biosensor data, such as physiological signals measured by a biosensor. The health state may indicate the physical, mental, and/or emotional health of the user. In some embodiments, the health state may be a level of stress of the individual user.

Health state information may be used in any suitable way. In some embodiments, a common representation of the health state may be displayed in the graphical user interface for each of multiple applications executing on or off the device on which health state information is gathered. Such a common representation may be a color scheme. The color scheme may be multiple colors corresponding to different health state levels. The multiple colors may represent a range of health state values determined from biosensor data or in any other suitable way. In such embodiments, the color scheme is applied to appropriate applications on the electronic device where the same color corresponds to the same health state level of a user. These health state levels may be absolute or relative health state levels. Absolute levels, for example, may correlate with specific degrees of physiological response above a baseline. In contrast, relative levels may correlate with a degree of physiological response above a baseline in comparison to a level that would be desirable in the user's context will at which the physiological response was observed.

Techniques described herein may be based on a component of system software executing on an electronic device passing health information to applications executing on the electronic device. This component may serve as a platform that enables applications that are coded to respond to health state information receive information needed to operate. In some embodiments, the health state information may include or information related to health state information such as an indication of a health state of an individual. Applications may access health state information through the platform by any suitable push and/or pull techniques. In some embodiments, an application may access information by the platform pushing the information to the application. In some embodiments, the application may access information by pulling or calling the information from the platform. The platform may pass the information in a notification or other push fashion, in which the platform may provide health state information to the applications without specific requests. Information may be pushed in this fashion as the health state information changes or periodically, such that each application may have health state information that is current. Updates of health state information, for example, may occur at a certain time interval, such as once per minute, or whenever a health state value changes by more than a threshold amount.

Any suitable form of interface may be used to support pushing or pulling of health state information. Some embodiments, the interface may support registration by applications programmed to receive health state information from the platform. Alternatively or additionally, the interface may support pulling of health state information from the platform. In such an embodiment, an application may query the platform to attain values of one or more health state parameters.

Health state information may be passed in any suitable format. For an example, if a common color scheme is used, the system software may pass the color value indicating a health state to the applications. The system software component may be the operating system, utility software, and/or any other component supporting application programming interface. An application may display such health information when displaying application information in an application graphical user interface. In such an embodiment, the application may pass the health state information with the application information to a display utility to control what information is displayed on a display of the electronic device. For example, a display utility may have an application programming interface that receives a color value in conjunction with information to be displayed. The application may pass the health state information, represented as a color, through this interface in conjunction with information generated by the application for display. However, it is not necessary that the application component pass the color value to the display utility. In other embodiments, the platform may supply the color component directly to the display utility. Such techniques may allow for an improved user experience by presenting health information within an application.

It should be appreciated that the foregoing description is by way of example only, and aspects of the invention are not limited to providing any or all of the above-described functionality, although some embodiments may provide some or all of the functionality described herein.

The aspects of the present invention described herein can be implemented in any of numerous ways, and are not limited to any particular implementation techniques. Thus, while examples of specific implementation techniques are described below, it should be appreciated that the examples are provided merely for purposes of illustration, and that other implementations are possible.

Techniques of the present invention are not limited to the type of health state information provided. A health state and/or health information for a user may be any particular status of the user's well-being. The health state may relate to the user's physical health, such as blood glucose level. Additionally, the health state may relate to the user's mental and/or emotional health, such as stress, depression, and/or anxiety level. It is recognized that an individual's overall health may be a combination of the physical, mental, and/or emotional health. Therefore, a combination of different aspects of health may be incorporated into applications on an electronic device using the techniques described herein. Although embodiments of the invention are described as relating to a user's stress level, this is a non-limiting example and it is recognized that any other suitable aspect of health may use the techniques described herein.

Any suitable sensor used for making physiological measurements of a user may be used. Such physiological measurements may include skin temperature, core body temperature, heat loss from the body, heart rate, pulse rate, beat-to-beat heart rate variability, skin conductance, oxygen, respiration rate, electrocardiography, electrodermal activity (EDA), galvanic skin response (GSR), electromyography, electroencephalography, electrooculography, blood pressure, hydration level, muscle pressure, activity level, body position, optical reflectance of blood vessels, oxygen saturation sensors, etc.

Additionally or alternatively, a sensor may measure the surrounding conditions and/or activity of a user. Such measurements may include ambient temperature or the user's surroundings and motion of the user, including three-dimensional motion. These additional measurements may be used in combination to physiological measurements to determine the health state of a user. Such additional measurements may provide context to isolate a health state from the physiological measurements, particularly when the physiological measurements may be influenced by the user's surroundings and/or activity. For an example, skin conductance measured for a user may be used to determine a stress level of the user. However, skin conductance may depend on the weather and/or activity level of the user, such as from exercising. By measuring the ambient temperature and/or motion of the user, identifying the stress level of the user from the skin conductance measurements may be improved and a more accurate detection of stress level may occur. It should be appreciated, however, that context information may be obtained in any suitable way. It could, for example, be acquired from an application, such as a calendar, running on a portable electronic device. Alternatively or additionally, the context information might be downloaded from a web service or obtained in any other suitable way.

Data from one or more physiological measurements may be collected over time by one or more biosensor devices. Physiological measurements may be collected continuously from the user's body. In some embodiments, physiological measurements may be collected at time intervals, such as 10 measurements per second. The time intervals may be designated by the user. In some embodiments, the time intervals may depend on a characteristic of measurement of the physiological signal performed by a biosensor. The time interval may be selected in order to acquire measurement statistics in order to determine a physiological state of a user. The time interval may depend on comparing a current physiological state to a threshold value. Such data may be used to determine the user's health, including physical, mental, and/or emotional state. The user may then use this information to track their health state over time. Accordingly, in some embodiments, a platform that receives physiological measurements may store and/or process those measurements to generate health state information. Such processing may entail correlating physiological data to an individual's health. For an example, specific physiological data may be known to correlate to an individual's stress level. By acquiring such specific physiological data over time, the user's stress status along a range of stress levels may be tracked over time. Such a range of stress levels may include a high stress level and a low stress level, such as may be characteristic of a calm and/or relaxed state. The user may use such stress level information to make decisions that may impact their overall well-being. In such embodiments, one or more of the biosensors described above and the analysis of the physiological data to determine a health state may be part of a biofeedback process. Alternatively or additionally, one or more applications may use such health state information to provide a form of biofeedback or to generate output that may impact a user's health state, such as by playing relaxing music or scheduling or recommending a nap or other low stress activity.

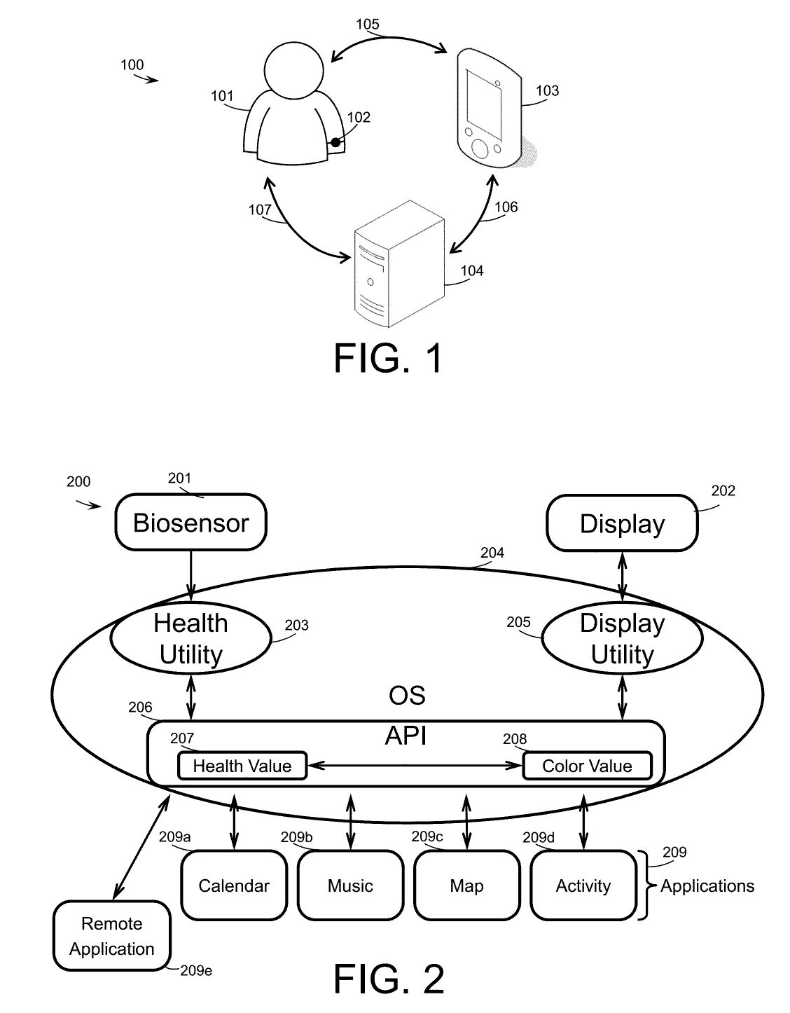

A schematic of a system incorporating a platform to provide health state information to one or more applications is illustrated by system 100 in FIG. 1. A user 101 may wear a biosensor device 102 to collect physiological measurements, including such measurements as described above. The biosensor device 102 may make any suitable physiological measurement or measurements, such as pulse rate and/or sweat response.

Although the biosensor is shown to be worn on the user's wrist in FIG. 1, any suitable type of biosensor may be located on or inside a user. The biosensor may be a component configured in a wearable article that suitable aligns the components with the wearer's anatomical structures. The biosensor may be integrated into a watch worn on the wearer's wrist. In some embodiments, the biosensor may be a bioband worn by the user on arm, wrist, leg, ankle, for example. In some embodiments, the biosensor may be a biopatch configured for placement in any suitable anatomical location on the user. In some embodiments, the biosensor may be a bioimplant and inserted under the user's skin to take physiological measurements. Additionally or alternatively, the biosensor may be worn by the user in any suitable way. The biosensor may be worn continuously by the user. In some embodiments, the user may periodically wear the biosensor device to acquire physiological measurements. Moreover, it is not a requirement that the biosensor be physically attached to the user. In some embodiments, the biosensor may gather physiological information without directly contacting the user. For example, a camera may take images of facial expression, complexion, eye movement, and/or limb movement as a way to determine a mental state. Sensor arrangements and techniques suitable for providing physiological data to determine health information may include components such as those described in U.S. patent application Ser. No. 14/702,208, filed May 1, 2015, and entitled “Wearable Electronics,” which is hereby incorporated by reference in its entirety.

Regardless of the form of the biosensor, data may be transferred between biosensor 102 and portable electronic device 103 via data link 105. Data link 105 may be implemented using any suitable technology, including wired or wireless technology. The biosensor data may be transferred in raw form, representing for example raw sensor readings. Alternatively or additionally the biosensor data may be processed before transfer, such that the transferred information indicates a health state or other processed information.

Portable electronic device 103 may be a smartphone and/or a tablet. However, any suitable hardware components may be used to receive and/or process physiological information from biosensor 102. A suitable device may include one or more hardware components, such as a processor, a display, memory, and/or a transceiver.

Data from the biosensor may be visualized on a display on the portable electronic device 103. The data may be directly displayed or may be processed before displaying by using one or more techniques as described herein. Additionally or alternatively, data from the biosensor may be transferred to a server 104 or other computing device separate from the portable electronic device via data transfer link 107. In some embodiments, server 104 may be a cloud computing service. Link 107 may be any suitable wired or wireless communication link and may entail, in some embodiments, both wired and wireless communication. For example, communication with server 104 may be performed in part over the Internet.

Server 104 may be used for data storage, data aggregation for multiple users, backup, and/or analysis. Data and analyzed results may be transferred between server 104 and portable electronic device 103. Data and analyzed results may be transferred between server 104 and portable electronic device 103 by data transfer link 106. Data transfer link 106 may be any suitable wired and/or wireless communication link. Such a link may entail processed results to be passed from portable electronic device 103 to server 104. Alternatively or additionally, link 106 may enable aggregated health status information, context information associated with health information, or other processed health status information to be passed from server 104 to portable electronic device 103, where the data may be further processed or displayed.

While only one user is shown in FIG. 1, biosensors may measure physiological information on more than one user at a time. In some embodiments, data collected for multiple users may be provided to the same portable electronic device 103 for processing and/or display. Alternatively or additionally, data from multiple users may be provided to the same server 104. Such data from multiple users may provide population health data and supply health statistics to determine a color scheme when representing a health state with a color value. The color may represent the health of the population or a selected subpopulation.

Alternatively or additionally, aggregated information may be used to determine a user's health state. An individual's health state may reflect where, relative to the aggregated information, the user's current state falls. For example, if the aggregated data shows that a subpopulation has become stressed during a time interval, a similar increase in the user's biosensor readings indicating stress may not lead to a proportionate increase in an assessment of that user. Conversely, if the user's stress level is above a statistical norm at a time when a selected subpopulation is below that statistical norm, the user's assessed health state may show a higher degree of stress. More generally, a user's assess health state may be computed based on a weighted average of comparisons of current sensed values to statistics of a plurality of aggregated values. Those aggregated values may allow comparisons to a population at large, a subpopulation deemed relevant to the user or prior values for the user.

In some such embodiments, physiological data from each user may be organized in a separate user account or otherwise kept separate to enable processing of health state information for each user separately. In other embodiments, the health state information may be aggregated to provide health state information about a population, such as a group of employees at a corporation. In those embodiments, health state information may or may not be associated with individual users. Additionally or alternatively, contextual information may be associated with the health state information about a population. The context information may include the individual's activity and/or location when the health information was acquired.

As part of understanding a user's overall well-being, or the overall well-being of a population of users, including their health, mental, and/or emotional state, additional information about the user and their life may be used to provide context to the physiological data and/or the analyzed results from such physiological data. Such additional information may include activities, events, sleep patterns, music, podcasts, phone calls, and user location. Such information may be derived in any suitable way, including from applications executing tasks for a user. An example of such an application is an application that can access e-mail for the user and email may be used to provide context to physiological data. In such embodiments, an application executing on portable electronic device 103, server 104, or other suitable computer device may access email messages and, using natural language processing techniques or other suitable processing, may derive an indication of an activity of a user, which may provide context to physiological data.

By combining physiological data with such contextual information, patterns, correlations, and/or trends of an individual's health state with respect to a specific user context may be observed. An individual's health state over multiple instances of the same type of activity may be compared. Through such an analysis, a pattern of an individual's health state with respect to a particular activity may be observed. When a biosensor is used to track a user's stress level and such physiological data is combined with contextual information, patterns may be observed to indicate certain conditions when a user may be more stressed or have a higher than normal stress level. For example, if an individual becomes more stressed during their morning commute, then physiological data will indicate a high stress level and the associated contextual information will indicate that the individual is on their morning commute. In this example, such contextual information may include user location information, time information, and/or date information. Data for the individual's health during multiple morning commutes may then be collected and analyzed. Such an analysis may indicate a pattern of an individual becoming more stressed during their morning commute.

Contextual information may be obtained in any suitable way. Individuals may use portable electronic devices as part of their daily activities for a wide range of tasks. Some non-limiting examples of tasks may include tracking and/or recording their schedules, listening to music, interacting on social media, browsing the Internet, playing games, finding locations and/or directions, and communicating via email, phone, video, and/or text message. By using a portable electronic device for such tasks, an individual may provide contextual information about their behavior and/or activities to the portable electronic device. Such contextual information may be collected and included in their applications such as email, calendar, phone (e.g., who was called and when), music, GPS information (e.g., where did the user go and when), and social media applications. Additionally or alternatively, a user may provide such contextual information to an electronic device through user input. Such user input may be provided within an application on a portable electronic device.

A platform as described herein enables integrating physiological data and/or analyzed health state of an individual with contextual information of the individual. Such techniques may allow for an improved biofeedback process in which a user is provided with information about their emotional state such that the user may control their emotional state and/or one or more applications access the emotional state information and generate output automatically that may impact the user's emotional state. As part of the biofeedback process, it may be beneficial to the user to visualize their health state information in conjunction with their contextual information. Such a visual representation may be displayed on portable electronic device 103. However, it should be appreciated that integrating health state information and context information may enable display of information and/or control of output for reasons other than to provide biofeedback. In some instances, a user's current health state may be displayed in a representation determined by a subset of aggregated health state information. Context information associated with health state information may be used to select such a subset.

In some embodiments, health state information may be processed or used by multiple applications on an electronic device. In such embodiments, it may be useful to the user to have a common representation of the user's health throughout the applications in which the health information is presented. By implementing a common representation, the user experience of interpreting the information may be simplified and/or easier because the user's health information is displayed using similar techniques across all applications.

Such common representation techniques may be used to display information in a graphical user interface on a display of the electronic device. In some embodiments, one or more colors and/or color schemes may be used as part of a common representation. In such a color scheme, a color may correspond to a specific health state. The color scheme may be a range and/or a gradient of colors. Such a range of colors may indicate different levels and/or states of health. In some embodiments, the color scheme may be a look-up table (LUT). In such embodiments, a particular health level and/or value may correspond to a specific color value in a LUT. In other embodiments, a range of health levels and/or values determined by a collection of aggregated health information may correspond to specific color values in a LUT. As an example, a range of stress levels of an individual may be represented by a color scheme. Such a color scheme may be a gradient of blue and red colors, where red may indicate a high level of stress and blue may indicate a low level of stress or a calm state. Any suitable components may be used for providing health state information to one or more applications. An example of a system in which health information from a biosensor may be displayed using a common representation is shown by block diagram in FIG. 2.

System 200 may include a biosensor 201, such as biosensor 100 described above in connection with FIG. 1. However, the specific form of biosensor 201 is not critical to the invention. FIG. 2 shows biosensor 201 providing physiological information to operating system 204. Operating system 204 may be an operating system of a portable electronic device, such as portable electronic device 103 (FIG. 1). However, it should be appreciated that operating system 204 may be any suitable software platform with which one or more applications may interact.

In the embodiment illustrated, operating system 204 includes a health utility 203. Health utility 203 may receive information from one or more sources and may generate health status information. Sources of information may include biosensor 201 or other biosensors. Alternatively or additionally, information processed by health utility 203 may include information from applications 209 or other components interfacing with operating system 204.

Physiological data from biosensor 201 may be provided to health utility 203. Such physiological data may be transmitted through wired communication and/or wireless communications, such as radio, wireless internet network, Bluetooth, and wireless USB. The physiological data may then be analyzed by health utility 203 to determine a health state. Such an analysis may include pre-established methods for determining a health state from physiological measurements. The health state may include one or more parameters that indicate the overall status of an individual's health. The health state may then be assigned a particular health value. Operating system 204 may support an application programming interface (API) to enable communication between components of operating system 204 and applications 209. The applications may be local applications executing on the same device on which operating system 204 is executing and/or maybe remote applications executing on a different computing device. Local applications 209a, 209b, 209c, and 209d are illustrated. Remote application 209e is also illustrated.

An application programming interface (API) may be used to transfer information between operating system 204 and applications 209. Any suitable programming techniques may be used to push or pull information through API 206 to allow applications to access information. The API may include routines, protocols, and tools for applications to interface with system and/or hardware components of the electronic device. The applications may access such routines and tools from the API as part of the application's programmed instructions. In the example illustrated in FIG. 2, API 206 enables applications 209 to access health state information from health utility 203 and to provide information to health utility 203. The information exchanged between health utility 203 and an application may include context information generated by that application and supplied to health utility 203. Health utility 203 may use such context information for one or more functions, such as to associate physiological information measured by biosensor 201 with a health state. Alternatively or additionally, health utility 203 may aggregate context information from multiple applications to provide higher-level context information to applications 209. In scenarios in which applications may collect or generate physiological information, that information may be provided through API 206 to health utility 203. API 206 may be configured using known programming techniques to facilitate exchange of such information.

In the embodiment illustrated in FIG. 2, health utility 203 may process physiological information from biosensor 201 and/or information from any other suitable sources to generate a health value 207. The health value may have any suitable form. In some embodiments, health value 207 may represent a value of a single health related characteristic. The value assigned may indicate the degree to which data associated with the user indicates that characteristic is present. For example the characteristic may be stress and the value may indicate a degree to which the user is stressed. In other embodiments, the health value may be indicate multiple user characteristics, such as stress and activity level.

In some embodiments, health state information may be represented as a color. The color may be selected based on a mapping from health value 207.

In some embodiments, an indexing operation may be used to relate a health value to a color value. Such indexing operations may allow for an application program to determine a color value corresponding to a particular health value and/or health state. In some embodiments a look-up table (LUT) may be used for relating a particular health value to a particular color value. However, it should be appreciated that other architectures may be used to distribute the operations depicted in FIG. 2 among the components. Such a LUT may be stored as a tool in API 206 and an application 209 may access the LUT by using the tool in API 206 such that the application receipts the health value and calls the tool when a color value is desired.

Color may be represented in any suitable way, including RGB values used in controlling computer displays. However, any suitable code may be used to represent a color. It should be appreciated that the mapping from health value 207 to color value 208 may be performed within API 206 or in any other suitable component. In some embodiments, API 206 may supply to applications only color value 208. In other embodiments, API 206 may supply only health value 207 and each application may map health value 207 to color used to display information. In yet other embodiments, API 206 may supply both health value 207 and color value 208. In such an embodiment, for example, an application may use color value 208 to control a display parameter while using health value 207 to control actions based on health state of a user.

Information provided through API 206 may serve as commands to components of operating system 204. As shown in the example of FIG. 2, operating system 204 includes a display utility 205 that may interact with display 202. Display 202 may be a display of a portable electronic device, such as portable electronic device 103 (FIG. 1). However, it should be appreciated that information as generated herein may be rendered on any suitable display. In the embodiment illustrated, a display utility 205 may be a display utility such as is included in operating systems as are known in the art. Such a display utility may receive information for display from executing applications and control the display to present that information. However, in other embodiments, display utility 205 may be a modified utility such that it receives health state information specifying display characteristics (such as color) and modifies information received from executing applications to visually present that information on the display modified according to the characteristic specified by the health state information.

Regardless of the specific form of API 206, an application that receives a color value and/or health information may use that information to control operation of the application and/or generate information for display. In embodiments in which multiple applications, such as applications receive color information, though each application generates different information, that information may be displayed in a common color scheme that varies based on the health state of the user.

Regardless of the specific approach used to generate health state information using a common representation, health state information may be provided through API 206 to one or more applications. These applications may use the health state information in selecting visual characteristics of information to be displayed as well as controlling operations of the application based on health state of the user. Further, some or all of the applications may generate context information that may be used for purposes such as to generate health values for control of operations performed by the applications. For context information that is generated by one application and used by another, the context information may be shared in any suitable way, including through procedure calls or other interface is implemented by API 206.

In the example of FIG. 2, applications 209 include local applications, which may include applications, whether now known or hereafter developed, to execute on a portable electronic device. In this example, local applications include a calendar application 209a, a music application 209b, a mapping application 209c and an activity-based application 209d. Examples of a calendar application 209a include an application that includes functionality of OUTLOOK or iCALENDAR productivity management software. Examples of a music application 209b include the functionality of ITUNES or MEDIAPLAYER applications. Examples of a mapping application 209c include the functionality of MAPQUEST or GOOGLE MAPS applications. Examples of an activity based application 209d include the functionality of NIKE+RUNNING application.

These applications, however, may differ from currently available applications in that they are configured to interact with API 206 such that they may receive health state information and may respond to that health state information. The applications may also be configured to provide context information through API 206. The specific use of health state information, and specific context information provided, may depend on the nature of the application. The context information provided by the applications may be used by API 206 to analyze the health state information and generate an indication of the health state information. In some embodiments, the context information may include information about a user at a time associated with health information of the user. The contextual information may include time, location, and/or activity that an individual is performing associated with the physiological data. Depending on the type of application, the context information may include a type of activity (e.g., listening to a song, exercising), a location, a time of day, an event, a meeting, a type of communication with a particular person. In this manner, the applications may provide details about the user's experiences in connection with the user's health state. For example, a music application may provide context information identifying a song the user was playing when health data is obtained by a biosensor. As another example, a mapping application may provide a location as context information at a time associated with health information.

Graphical user interfaces rendered by the applications may present health information in a manner such that the health information is connected with context information. In some embodiments, health information may be portrayed in a color scheme and colors associated with particular visual elements within a graphical user interface may correspond to specific health states either for an individual and/or a population having a plurality of individuals. For example, calendar application 209a may use health state information to select a color for display of blocks of time depicted on the calendar. Context information provided by a calendar application may include scheduled activities during a period of high or low stress. Music application 209b may use health state information to select times at which music is played and/or specific songs that are played. Mapping application 209c, for example, may use health state information to designate locations on a map in which a user has exhibited high or low stress by coloring those map locations in accordance with the health state information. The mapping application may also provide as context information identification of locations in which the user has exhibited high or low stress. Activity application 209d, for example, may use health state information to color a user interface to signal a greater or lesser need for a user to exercise.

Remote application 209e may be any suitable application, including one that performs any of the functions described for the local applications. For example, a calendar application may be run locally on a portable computing device or may be executed “in the cloud” and accessed over a network. However, in some embodiments, a remote application 209e may perform functions or access data not available locally on user computing device.

For example, remote application 209e may receive health state information from a multiple computing devices associated with the same user at different times or associated with different users. As a result, remote application 209e may aggregate health state information and display or provide control based on that aggregated information.

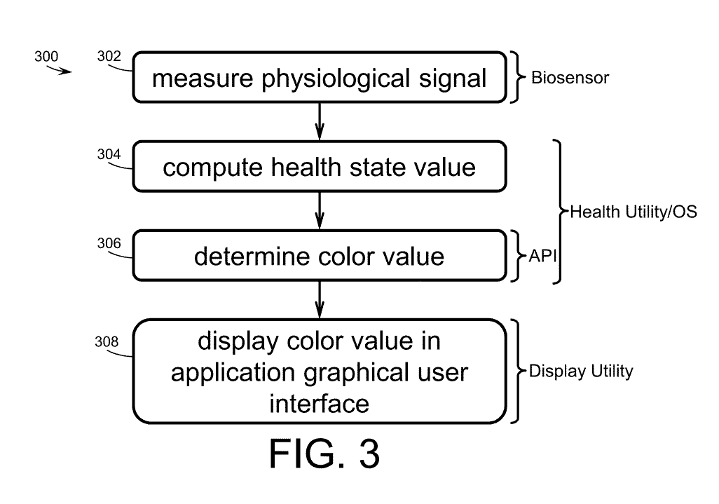

It should be appreciated that the architecture for processing physiological data is exemplary and not limiting. Physiological data may be converted into a color value representing a particular health state in any suitable way. FIG. 3-5 are flowcharts of exemplary methods, with corresponding indications of hardware and/or software that performs steps in a method.

In the example of FIG. 3, process 300 begins at block 302 where a physiological signal of a user may be measured by a biosensor. Data pertaining to the physiological signal may be sent to a health utility (such as health utility 203) within the operating system of a device.

At block 304, the health utility may compute a health state value. Any suitable computation technique may be used to compute a health state value from a physiological signal, including techniques described in copending application U.S. Patent Application 2011/0245633 with Ser. No. 13/040,816, filed Mar. 4, 2011, and entitled “Devices and Methods for Treating Psychological Disorders,” which is hereby incorporated by reference in its entirety. However, the computation at block 304 may include context information or other information instead of or in addition to the physiological signal. In the embodiment illustrated, health state of the user is indicated by a single value, which may represent stress or other health parameter. In other embodiments, however, different or additional health parameters may be reflected in the computed health state value.

At block 306, a color value may be determined from the health state value. In the illustrated embodiment, this determination is made by the health utility. Additionally or alternatively, the health state value may be sent to an application programming interface that may use the health state value to determine a color value 306. Regardless of the specific component that determines the color value, the color value may be used to select display characteristics in a graphical user interface for one or more applications. In block 308, a display utility that manages a display may apply the color value to set the color of one or more items generated by an application for display. The color value may be supplied to the display utility in any suitable way. In some embodiments, the color value may be passed to the application through on API such that the application may associate color value specific objects to appear on the display. Alternatively or additionally, the color value may write directly on the display. Display utility may apply the color value to modify display characteristics in one or more objects. However, the color value may be used in any suitable way, such as a default color or as a background color. In embodiments in which the color value is sent to a display utility, the display utility may apply this color value to information for display generated by multiple applications. In this way, a common color scheme, in which color is associated with health state, may be implemented for user interfaces of multiple applications.

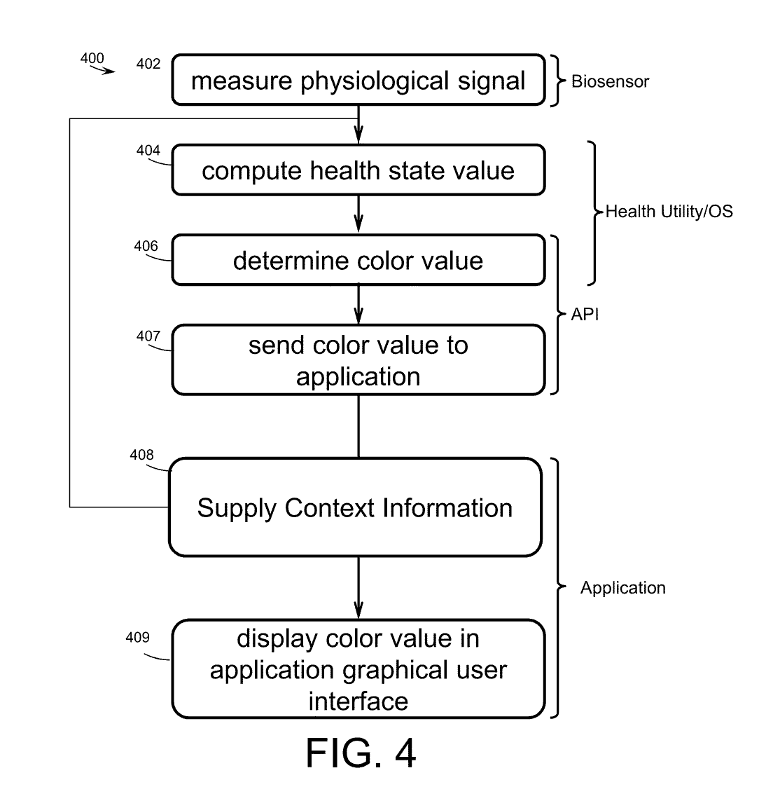

As another example of the manner in which color information may be used, FIG. 4 illustrates an alternative process 400. In the example of FIG. 4, a physiological signal of a user may be measured by a biosensor at block 402. Data associated with the physiological signal may be transferred to an operating system of an electronic device, such as to a health utility within the operating system installed on an electronic device. At block 404, such a health utility may compute a health state value based at least in part on the measured physiological signal. In this example, processing at block 404 receives as input context information. Accordingly, the computation of the health state value may be based on the physiological signal as well as pass context information.

The computed health state value from block 404 may be used to determine a color value by block 406. Such a color value may be determined by any suitable component, such as an operating system, health utility, and/or API of an electronic device.

In process 400, the color value determined at block 406 may be sent to one or more applications at block 407. This act may be performed by an API. Though, in other embodiments, any other suitable component may be used to distribute health state information.

Blocks 408 and 409 represent actions within an application that interfaces to an API that supports exchanging health state information and context information. In block 408, the application may supply context information to the health utility, which may use this as described above in connection with block 404 or in any other suitable way. At block 409, the application may select objects for display on its user interface to appear in a color represented by the color value. The application may transfer both application information representing the objects to be displayed and the color value to a display utility. The display utility may display the color value in an application graphical user interface. When multiple applications receive the same color value, those applications can present objects with a common color. In some embodiments, the selected objects for each application may be objects that have a connection with the health state of the user such that objects having a connection to health state, regardless of the application that generates them, can have a common color. The objects may have a connection to health state, for example, by depicting or representing operations performed while the user is projected to be in the health states represented by the color.

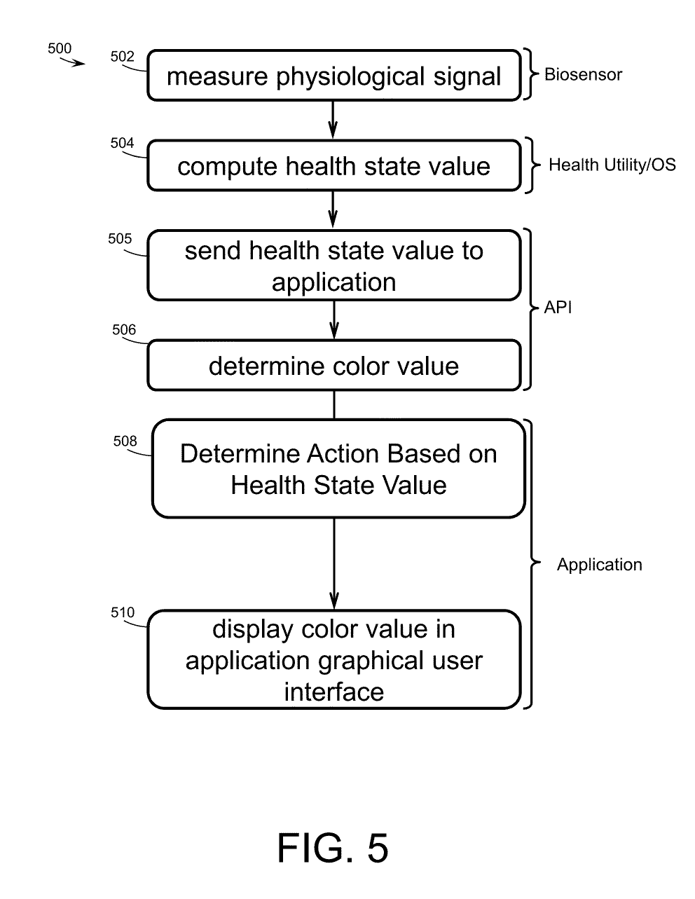

FIG. 5 outlines process 500, representing a further variation of a approach for generating and using health state information. Process 500 may begin at block 502 where a biosensor may measure a physiological signal of a user. Data from the measurement at block 502 may be transferred to a health utility, which may be within an operating system of an electronic device.

At block 504, the health utility may compute a health state value. The computed health state value may be sent to one or more applications via API at block 505. As noted above, an application programming interface may operate on a push or a pull model such that sending the data at block 505 may occur in response to a request for data from the application or may be triggered in any other suitable way.

At block 506, the determined health state value may be mapped to a color value. In the example of FIG. 5, this mapping is performed within an API. For example, an application may place a call on the API to determine a color value corresponding to the health state value computed at block 504.

In this scenario, the application has access to both the health state value and the color value. Accordingly, the application may generate information for display in the designated color. The application may also use the health state value for controlling one or more operations. Accordingly, at block 510, the application may instruct a display utility to display application information, including one or more objects, using the color value in an application graphical user interface.

Alternatively or additionally, at block 508, the application may determine one or more actions based on the health state value. These actions may be any of the actions described above or any other suitable actions that may be impacted by or may impact health state.

Health information from one or more populations of people may be used to determine color information. A selected population may include people having one or more characteristics such as age, biological sex, geographical location, and/or occupation. The health data from a selected population may then be used determine the manner in which color information displays a user's health information. In some embodiments, the health statistics from the population may be used to determine a color scheme that is then used to determine a color value. In other embodiments, contextual information may be used to select a population that is then used to normalize the user's health state value prior to determining a color value.

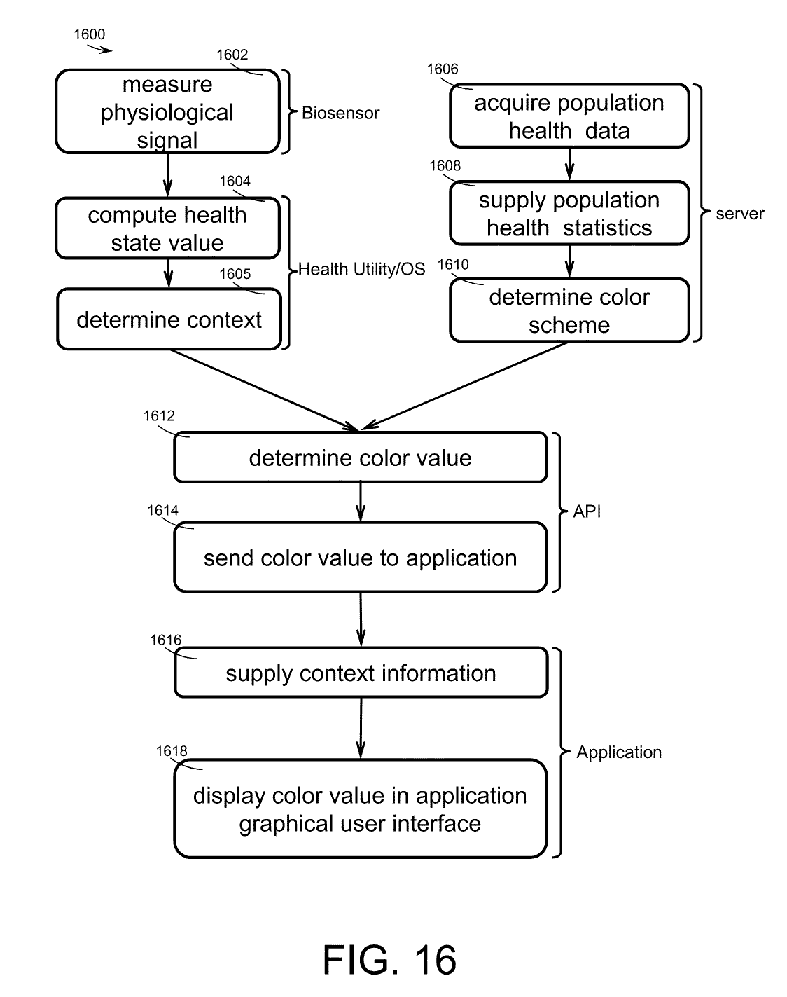

As an example of the manner in which population health data may be used to determine color information, FIG. 16 illustrates process 1600. In the example of FIG. 16, a physiological signal of a user may be measured by a biosensor at block 1602. Data associated with the physiological signal may be transferred to an operating system of an electronic device, such as to a health utility within the operating system installed on an electronic device. At block 1604, such a health utility may compute a health state value based at least in part on the measured physiological signal. In this example, processing at block 1605 determines context information and may pass the context information along with the computed health state value. Such context information may be supplied by an application and received by the health utility that is part of the operating system.

Data associated with population health data may be acquired on a server at block 1606. Such data may include health information from other users. The server may be a located locally or remotely, such as a cloud server. The population health data may represent historical data acquired over time from one or more users. The population health data supplies population health statistics at block 1608. The health statistics may indicate statistical information determined from health state values associated with the population. Such statistical values may include a range of health state values, average health state value, and/or standard deviation. Statistical information may change over time as additional health data of the population is acquired. Such statistical information may be used to determine a color scheme at block 1610. As an example, when a range of health state values is included in the statistical information a color scheme may be selected that covers that range of values where individual colors indicate health state values within the range of values.

The computed health state value from block 1604, the determined context information from block 1605, and the color scheme from block 1610 may be used to determine a color value by block 1612. Such a color value may be determined by any suitable component, such as an operating system, health utility, and/or API of an electronic device.

In process 1600, the color value determined at block 1612 may be sent to one or more applications at block 1614. This act may be performed by an API. Though, in other embodiments, any other suitable component may be used to distribute health state information.

Blocks 1616 and 1618 represent actions within an application that interfaces to an API that supports exchanging health state information and context information. In some instances, the application may supply context information to the health utility, which may use this as described above in connection with block 1605 or in any other suitable way. At block 1618, the application may select objects for display on its user interface to appear in a color represented by the color value. The application may transfer both application information representing the objects to be displayed and the color value to a display utility. The display utility may display the color value in an application graphical user interface. Since health data of a population was used to determine the color scheme, the color value may designate the user's health state value relative to health statistics of the population. In some instances, the color value may indicate the user's health state relative to an average health state of the population providing the health statistics. For example, the color value may indicate whether the user's health state is average, below average, or above average compared to the health of the population.

When multiple applications receive the same color value, those applications can present objects with a common color. In some embodiments, the selected objects for each application may be objects that have a connection with the health state of the user such that objects having a connection to health state, regardless of the application that generates them, can have a common color. The objects may have a connection to health state, for example, by depicting or representing operations performed while the user is projected to be in the health states represented by the color.

In some instances, contextual information may be used to select the population, and thus the health data, used to determine a color value for the user's health state value. Characteristics of the individuals are associated with the population health data. Such characteristics may include contextual information of the individuals, such as time of day, individual's current activity, previous activity, and/or geographical location. Characteristics of the individuals in the population may be used to select a subset of the population based on the contextual information of a user. Health data associated with the selected subset population may be used to determine a color value for the user's health state value. For example, contextual information that indicates a user is commuting to work may be used to select a subset population of individuals that have a similar commute schedule as the user. Health data of the similar commuters may be used to determine a color value for the health state value of the user. As an example, the user's health state value may be normalized to the health statistics of the subset population, in this case the similar commuters.

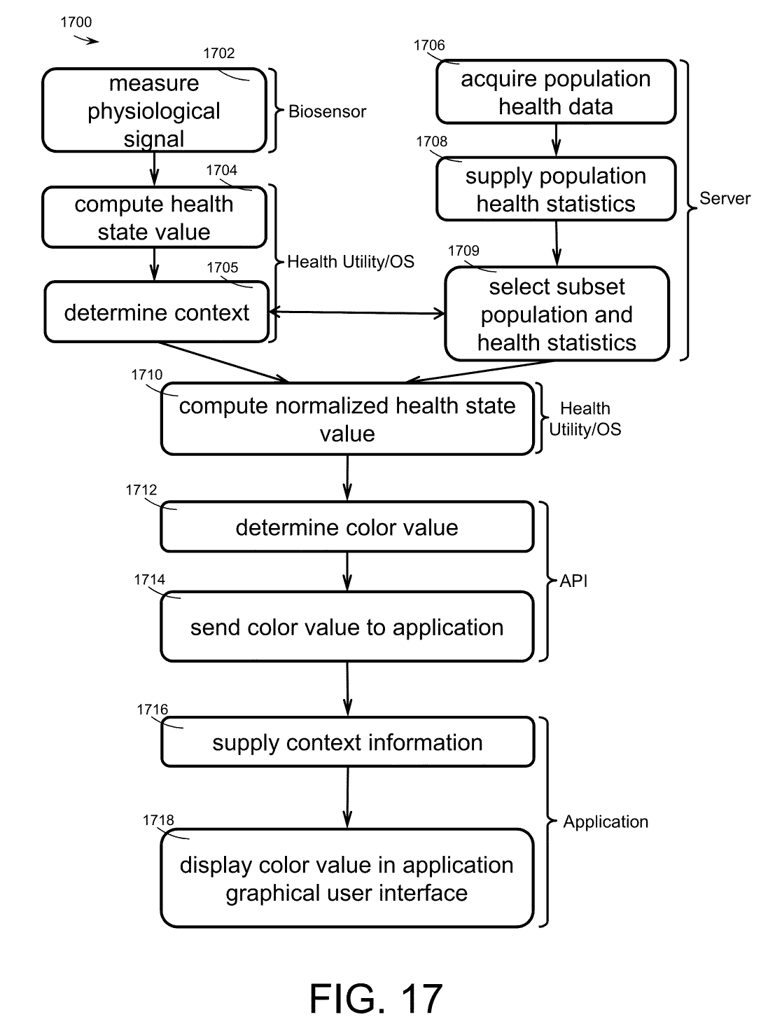

As an example of the manner in which contextual information may be used to select a subset population as part of determining a color value to represent a user's health state is process 1700 illustrated in FIG. 17. In the example of FIG. 17, a physiological signal of a user may be measured by a biosensor at block 1702. Data associated with the physiological signal may be transferred to an operating system of an electronic device, such as to a health utility within the operating system installed on an electronic device. At block 1704, such a health utility may compute a health state value based at least in part on the measured physiological signal. In this example, processing at block 1705 determines context information and may pass the context information along with the computed health state value. Such context information may be supplied by an application and received by the health utility that is part of the operating system.

Data associated with population health data may be acquired on a server at block 1706. Such data may include health information from other users. The server may be a located locally or remotely, such as a cloud server. The population health data may represent historical data acquired over time from one or more users. The population health data supplies population health statistics at block 1708. The health statistics may indicate statistical information determined from health state values associated with the population. Such statistical values may include a range of health state values, average health state value, and/or standard deviation. Statistical information may be updated over time as additional health data of the population is acquired. A subset of the population may be selected at block 1709 to provide selected health statistics. Contextual information determined by block 1705 may be used in selecting the subset population at block 1709. Contextual information may be sent from the health utility to the server and the subset population may be selected based on the contextual information provided. Relevant health statistics for the selected subset may be retrieved and passed to the health utility of the operating system. In this manner, the contextual information may be used as part of instructions for retrieving health statistics relevant to the contextual information provided.

The statistical information of the health of the subset population may be used to normalize the health state value of the user at block 1710. As an example, the subset population may provide a range of health state values and the user health state value computed by block 1704 may be normalized by the range of values. Once the health state value is normalized, the color value may be determined by block 1712.

The computed normalized health state value from block 1710 and/or the determined context information from block 1705 may be used to determine a color value by block 1712. Such a color value may be determined by any suitable component, such as an operating system, health utility, and/or API of an electronic device.

In process 1700, the color value determined at block 1712 may be sent to one or more applications at block 1714. This act may be performed by an API. Though, in other embodiments, any other suitable component may be used to distribute health state information.

Blocks 1716 and 1718 represent actions within an application that interfaces to an API that supports exchanging health state information and context information. In some instances, the application may supply context information to the health utility, which may use this as described above in connection with block 1705 or in any other suitable way. At block 1718, the application may select objects for display on its user interface to appear in a color represented by the color value. The application may transfer both application information representing the objects to be displayed and the color value to a display utility. The display utility may display the color value in an application graphical user interface. Since health data of a population was used to normalize the user's health state value, the color value may designate the user's health state value relative to health statistics of the subset population. In some instances, a range of color values may designate the range of health states for the subset population. The color value displayed may indicate the user's health state relative to the range of health states for the subset population. For example, the color value may indicate whether the user's health state is average, below average, or above average compared to the health of the subset population. The user's normalized health state value may differ depending on the subset population selected. Different subset populations may provide different health statistics used in the normalization process of the health state value. As a result, different normalized health state values can be obtained by using health statistics from different subset populations.

When multiple applications receive the same color value, those applications can present objects with a common color. In some embodiments, the selected objects for each application may be objects that have a connection with the health state of the user such that objects having a connection to health state, regardless of the application that generates them, can have a common color. The objects may have a connection to health state, for example, by depicting or representing operations performed while the user is projected to be in the health states represented by the color.

In some instances, historical health data previously acquired from the user may be used to determine a color value. This historical health data may change over time as additional user health data is acquired. A user's historical health data may be used to normalize a user's current health state value by providing statistical information of the historical health data. Contextual information of the user obtained when the health data is acquired may be stored associated with the health data. The historical context data may be used to identify particular health data obtained under similar contexts. The current context of the user may be used to select a subset of the historical health data obtained based on the contextual information associated with the historical health data. Within the subset of health data, the contextual information may reflect one or more similarities of the user's context. Statistical information of the subset of health data may be computed and used to normalize a current health state value of the user. Using such a technique, a current health state value may be normalized based on previously acquired user health data obtained under a similar context as the current context of the user. The current state health value of the user becomes normalized based on the historical health data of the same user. As an example, contextual information may include an amount of the user slept the night before. As health data is acquired and stored, this sleep information is also acquired and stored in association with the health data. A current health state value and current sleep amount may be obtained and statistical information of the user's historical health data when the user has slept a similar amount may be used to normalize the current health state value. For example, if the user slept 7 hours the night before, then historical health data obtained when the user also slept around 7 hours may be used to normalize the current user's health state value. The normalized health state value may be used to determine a color value to reflect the current health of the user.

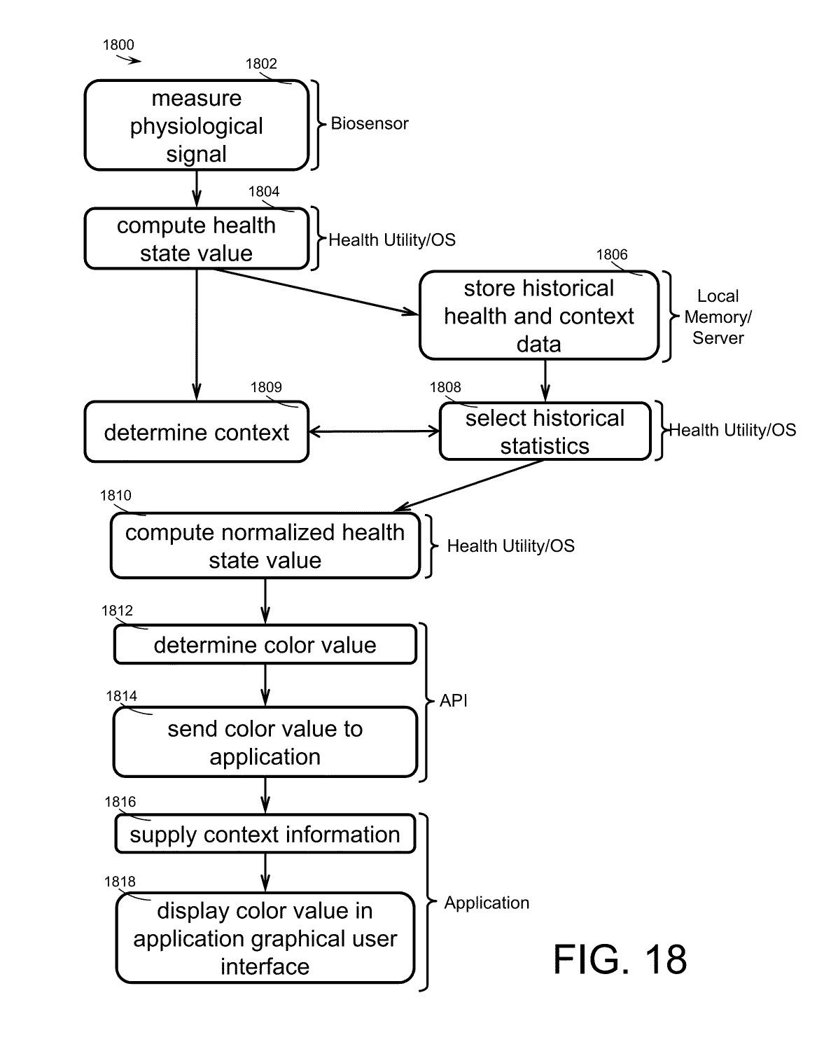

As an example of the manner in which contextual information may be used to select historical health data as part of determining a color value to represent a user's health state is process 1800 illustrated in FIG. 18. In the example of FIG. 18, a physiological signal of a user may be measured by a biosensor at block 1802. Data associated with the physiological signal may be transferred to an operating system of an electronic device, such as to a health utility within the operating system installed on an electronic device. At block 1804, such a health utility may compute a health state value based at least in part on the measured physiological signal. In this example, processing at block 1809 determines context information and may pass the context information along with the computed health state value. Such context information may be supplied by an application and received by the health utility that is part of the operating system.

As health state values are acquired, they are stored along with data indicating the context of the health state values. In this example, data associated with historical health and context data may be acquired on a server at block 1806. The server may be a located locally or remotely, such as a cloud server. The historical health and context data is acquired over time for the user. The health data supplies health statistics at block 1808. The health statistics may indicate statistical information determined from health state values associated with the population. Such statistical values may include a range of health state values, average health state value, and/or standard deviation. Statistical information may be updated over time as additional health data of the user is acquired. A subset of the health data may be selected at block 1808 to provide selected health statistics. Contextual information determined by block 1809 may be used in selecting the health data and statistics at block 1808. Contextual information may be sent from the health utility to the server and the subset of the health data may be selected based on the contextual information provided by using the historical contextual information previously acquired. Relevant health statistics for the selected subset may be retrieved and passed to the health utility of the operating system. In this manner, the historical contextual information may be used as part of instructions for retrieving health statistics relevant to the current contextual information provided.

The statistical information of the health of the subset population may be used to normalize the health state value of the user at block 1810. As an example, the historical health statistics may provide a range of health state values and the user health state value computed by block 1804 may be normalized by the range of values. Once the health state value is normalized, the color value may be determined by block 1812.

The computed normalized health state value from block 1810 and/or the determined context information from block 1809 may be used to determine a color value by block 1812. Such a color value may be determined by any suitable component, such as an operating system, health utility, and/or API of an electronic device.

In process 1800, the color value determined at block 1812 may be sent to one or more applications at block 1814. This act may be performed by an API. Though, in other embodiments, any other suitable component may be used to distribute health state information.

Blocks 1816 and 1818 represent actions within an application that interfaces to an API that supports exchanging health state information and context information. In some instances, the application may supply context information to the health utility, which may use this as described above in connection with blocks 1806 and 1809, or in any other suitable way. At block 1818, the application may select objects for display on its user interface to appear in a color represented by the color value. The application may transfer both application information representing the objects to be displayed and the color value to a display utility. The display utility may display the color value in an application graphical user interface. Since historical health data of the user was used to normalize the user's health state value, the color value may designate the user's health state value relative to health statistics of the user. In some instances, a range of color values may designate the range of health states for the user. The color value displayed may indicate the user's health state relative to the range of health states that the user has previously experienced. For example, the color value may indicate whether the user's health state is average, below average, or above average compared to the user's previous health. The user's normalized health state value may differ depending on the context information used to select the subset of the historical health data to provide statistics. Different subsets of the historical data may provide different health statistics used in the normalization process of the health state value.

When multiple applications receive the same color value, those applications can present objects with a common color. In some embodiments, the selected objects for each application may be objects that have a connection with the health state of the user such that objects having a connection to health state, regardless of the application that generates them, can have a common color. The objects may have a connection to health state, for example, by depicting or representing operations performed while the user is projected to be in the health states represented by the color.

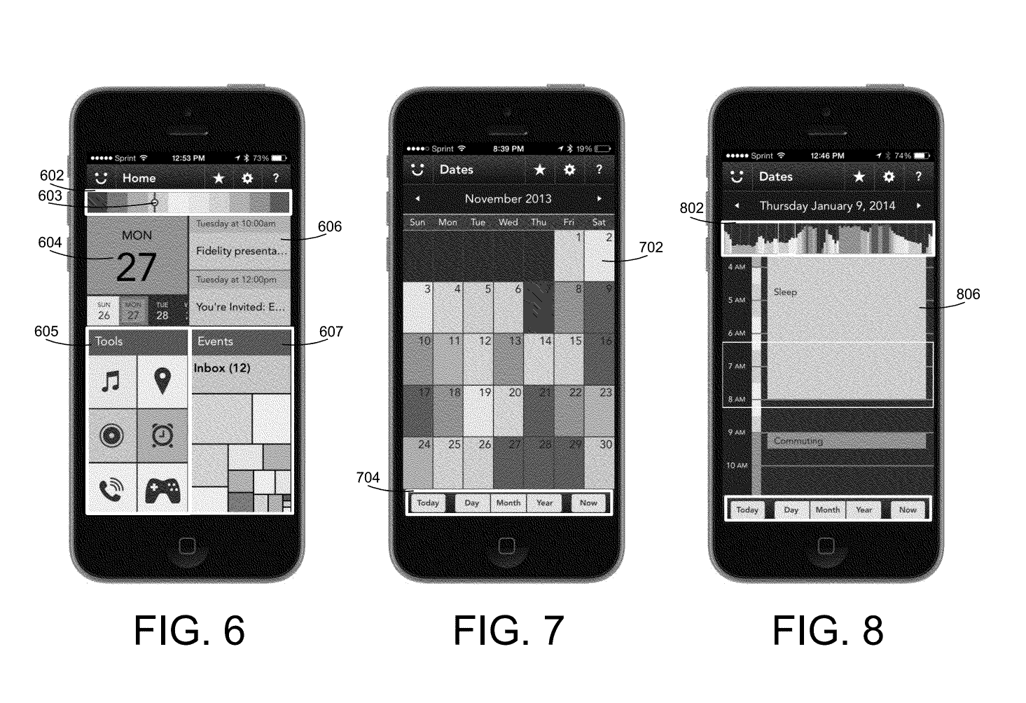

Health information may be processed in a variety of applications. Examples of non-limiting applications may include applications that manage for a user a calendar, events, music, a map, a phone, text messaging, video messaging, and/or games. Regardless of the type of application, health information may be presented in one or more application graphical user interfaces by a common representation using the above-described techniques. FIGS. 6-14 are examples of application graphical user interfaces for a variety of applications where the common representation of health state information is a color scheme. In these examples, the color scheme consists of different hues of blue and red where the two extremes are dark blue and dark red while the middle two hues are a light blue and a light red. In such a color scheme applied to a user's health state, extreme health states may be represented by dark blue and dark red and more moderate health states represented by a light blue and/or light red. Although blue and red colors are used in these examples, any other color scheme may be used as a common representation. Moreover, hue is used in these examples to graphically illustrate a magnitude of a health state. In some embodiments, the hue may graphically illustrate a magnitude of a health state of a user relative to health data of a population. Other characteristics of color may alternatively or additionally be used, such as colorfulness, chroma, saturation, lightness, or brightness.

Additionally or alternatively, another type of visual representation may be used instead of color as a common representation. Such other types of visual representation may include symbols, patterns, gradients, and/or text.

FIG. 6 illustrates a home screen of an electronic device having a color scheme corresponding to a user's health state for different objects displayed by different applications. In this example, the electronic device is a portable electronic device, such as a smart phone. The different colors represented on the screen are used to represent a different health state associated with some aspect of the application. Color bar 602 indicates a range of colors associated with different health states. Dark blue is on the left of the screen and dark red is on the right of the screen within color bar 602. Although ten colors are shown in color bar 602 any suitable number of colors may be used to indicate a range of health states of a user. Such a color bar may be used when a value for health state is selected from an enumerated set of values, such as a set of one of 10 possible values, so that each indicated color corresponds to a possible value of health state. In other embodiments, the displayed colors may be presented as a continuum, corresponding to a continuous range of possible values for health state. However, it should be appreciated that there need not be a 1:1 relationship between displayed colors and health states. For example, a number of discrete colors may be displayed even if there is a continuous range of possible values for health state.

Indicator 603 may indicate a health state of the user at some time. In some embodiments, indicator 603 may represent a current health state of the user. Indicator 603 may be any suitable shape and/or shapes. Moreover, it should be appreciated that in some embodiments, indicator 603 may, instead of providing output to a user, may be an active display element that enables user input. For example, in some embodiments and/or in some scenarios, indicator 603 may be configured to be adjustable by a user, allowing a user to specify a color, which is associated with a health state. That health state designated by positioning of indicator 603 may serve as a target or desired health state. However, it should be appreciated that input designating a health state may be used in any suitable way, such as a threshold health state or a health state as perceived by the user. Applications configured to be health state aware may use this information to provide biofeedback to guide the user to the target health state or to keep the user's health state above or below the threshold, as appropriate for the meaning of the input. Alternatively, an input of the user's perceived current health state may serve as a check or may be used for refining computations of current health state based on physiological data and/or context information.

The home screen may include health information associated with applications, such as a calendar. Graphic 604 indicates a particular color value representing a health state of the user associated with a calendar day. The color value in graphic 604 may represent an average health state of the user throughout that day. Such an average health state may be based on past and/or current physiological measurements. In such embodiments, the color value of graphic 604 may change as the health state of the user changes and additional physiological measurements are acquired to indicate a change in health state.

In some embodiments, the color value in graphic 604 may be based on predicting future health states of the user. The color value, for example, may indicate a health state associated with activities scheduled on the user's calendar for a specific day. In other embodiments, the color value in graphic 604 may be a combination of past, current, and/or predicted health states of a user.

The home screen may also include objects, each corresponding to an event as shown in graphic 606. These events may be past, present, or future, and the color associated with each object may indicate measured or projected health state while the user is engaged in the event.

The home screen may also include objects generated by other applications, such as objects 605. The object may be grouped by function associated with the applications, which in this example is that the objects are indicated as tools. These applications may play music or other audio media or provide maps, a clock, alarm, a phone, and/or games. Each application may designate a color value for a health state associated with that application. The health state may be based on the current health state of the user or may represent other health state such as indicating a historical health state, a predicted health state, a deviation from a target or a threshold health state.

For example, an object included among objects 605 may be generated by an alarm clock application. The health state associated with that application may indicate a projected health state based on the amount of sleep the user is likely to get based on the set time for the alarm. The set time for the alarm in combination with context information, such as activity level indicating when the user typically falls sleep, may be used by the alarm application, or any other suitable component, to project a health state. The icon representing the alarm may be depicted with a color indicating this health state.

In some embodiments, the objects included among objects 605 may represent sub-categories of applications. An object representing a sub-category may be accessed by the user to bring up a further menu presenting objects associated with applications within the sub-category. For example, one of the objects may be associated with the sub-category of games. Selecting that object may access a collection of objects associated with individual games. Each of those objects may be presented with a color code representing a health state associated with the individual games.

The object illustrated on the home screen representing the subcategory may represent an aggregate health state for applications within the subcategory. An aggregate health state may be determined in any suitable way. For example, in embodiments in which health states are represented by numeric values, that medical averaging may be used to determine an aggregated health state. In other embodiments, in which health state is represented by an enumerated set, which need not contain numeric values, the aggregated health state may represent a mode or other statistical function of the values of the applications in the subcategory.

Any suitable approach may be used to associate health states with applications. It should be appreciated that the approach used to associate health states with applications may be different for different ones of the applications. Moreover, a user interface may present colors associated with applications in different ways at different times. In some embodiments, the user interface includes a control which enables a user to specify the significance of colors at any time. As an example, a color value may represent a health state of the user when an application is active and/or being used by the user. At other times or in other scenarios, a color value may represent a projected health state that may be generated by the user accessing the application. Alternatively or additionally, the color value may represent a health state acquired on prior occasions when the user accessed the application. In some embodiments, the prior occasions may be filtered in some way such that the color value represents an aggregate of a subset of prior occasions. The filtering criteria may be specified by a user through the user interface or in any other suitable way.

A color values of objects 605 may represent an aggregate health state for a sub-category based on previously acquired physiological measurements when a user is associated with one or more applications in the sub-category. Additionally or alternatively, a color value of a sub-category in graphic 605 may be the most recently measured health state of the user associated with the sub-category. Similar color values may be used to represent sub-categories of events in objects 607 and will be discussed in more detail in reference to FIG. 13.

It should be appreciated that an application may generate objects for display in more than one format and that color values may be used to indicate health state in some or all of the formats. As shown in FIG. 6, calendar information may be depicted by objects each representing a day or an event in a list.

FIGS. 7-9 illustrate alternative ways in which calendar information may be depicted. Nonetheless, in each of these examples, objects generated by the calendar application may be color-coded based on health state information associated with a time range or activity depicted in the presentation of information. For example, FIG. 7 illustrates a control bar 704 which may allow a user to specify granularity with which information is presented. In this example, the granularity options are “day”, “month” or “year”. Depending on the granularity selected, the objects displayed may differ. Nonetheless, each object displayed may be color-coded to present health state information associated with that object.

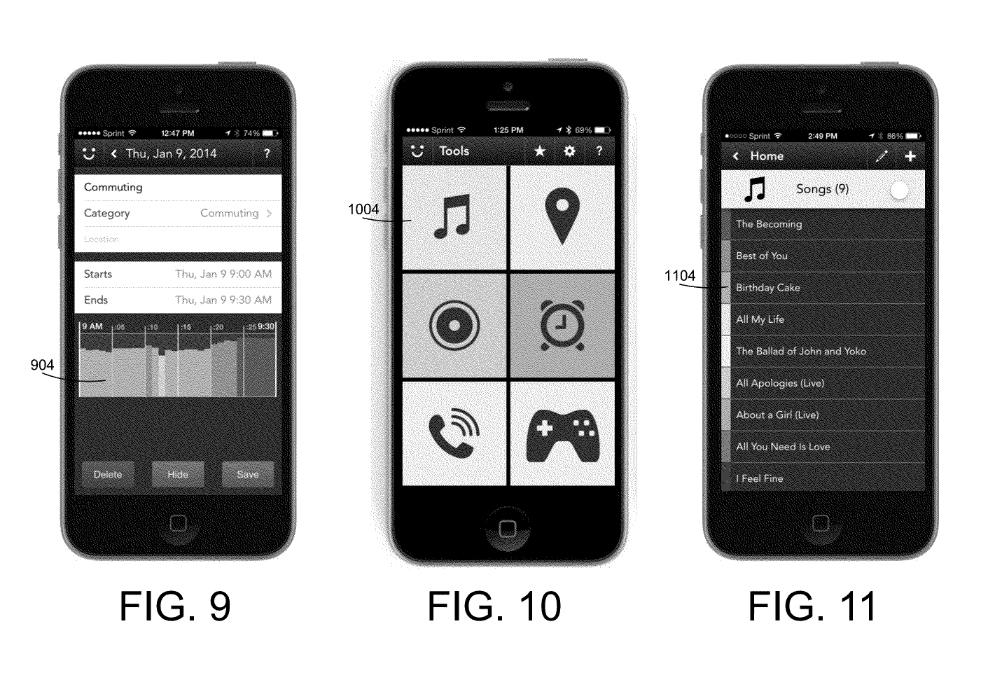

FIG. 7 illustrates calendar information displayed with the granularity of a month. Each object in that display may represent a day, each of which is color coded as other examples, the color coding may indicate a measured, current and/or projected health state. FIG. 8, in contrast represents granularity of a day. In that example, each object may represent an event scheduled for a time during the day. Time may be divided intervals, which are half-hour blocks in the example of FIG. 8. FIG. 9 shows an even more detailed view of information, identifying a scheduled event, and showing, in histogram 904, health state information collected during that event. In this example, health state information is shown for time intervals of 1 minute. Interfaces presenting higher or lower levels granularity may alternatively or additionally be provided. For example, though not illustrated, a display view showing events over one year may be selected. The objects presented in that view may represent months of the year with colors indicating health state information used to depict each of those months.

In the illustrated example, the granularity may be selected using known input techniques, such as pressing buttons on control bar 704. However, it should be appreciated that any suitable navigation commands may be used to move between different views of calendar information. In some embodiments, the display format may be specified by a user based on a swipe command on a touchscreen of the device presenting a graphical user interface. In some embodiments, a velocity of the swipe may correlate with a degree of granularity information is presented.

For example, a user interface driver within the device operating the touchscreen may detect the swipe velocity. Swipe velocity information may be provided to the calendar application, which may respond by altering the format of the information for display. In some embodiments, a slow swipe may be interpreted as an indication for fine granularity, such that information may be presented for portions of an hour or minutes as indicated in FIG. 8 or 9. In such embodiments, a faster swipe may indicate a granularity of days or months, such as is indicated in FIG. 7. However, the specific meaning of fast or slow may be different for different embodiments. For example, in some embodiments a faster swipe may indicate a finer granularity.

Regardless of the granularity of information displayed by a calendar application, the information may be presented in varying colors to represent health state information. FIG. 7 shows a month view 702 of a calendar application where each day may have a color value associated with health information of a user for that day. Such health information may be an average health state of the user throughout the day. By using such an application user interface, a user may readily see how their health changes daily within a selected month. A calendar application may also have other calendar views such as, today, day, and year view which may be selected by section 704 of the application graphical user interface. FIG. 8 shows a day or daily view of health information of a user. Such a daily view may include a profile of the health state of the user at different moments throughout a day, such as in graphic 802. Such a user interface may include an object, such as object 806, representing an activity of the user. Such an object may be presented with a color indicating health state during or as a result of that activity. FIG. 9 similarly shows a user interface, with different level of granularity, with activities similarly illustrated in color to represent health state information. In some embodiments, the colors used to represent health state information may be in a common format, regardless of the level of granularity or other characteristics of the user interface being presented.

FIG. 10 is an example of another user interface, which may be displayed when a user accesses the tools object 605 (FIG. 6). Here, icons representing applications are presented. The background color for each of the icons may be selected based on health state information. As a result of the use of a platform that provides health state information to multiple applications, the color scheme used to represent various health states may be the same for the user interface of FIG. 10 as it is for a user interface presented by other applications.

The common color scheme may be carried to user interfaces for other applications. For example, FIG. 11 illustrates a user interface presented by an application that controls playing of songs. In this example, song titles in a list from which a user may select songs are tagged with objects, such as object 1104, that associates a color with song titles.

Providing a common color scheme for objects on a display provides information that enables multiple functions to be performed. For example, the applications may be used as part of the biofeedback process. The user may readily see how the state of their health correlates to specific events and/or activities. The user may then choose to change their behavior and/or surroundings based on this information in order to improve their overall health. For an example, the individual may observe that for particular songs their stress level is reduced. Such information may then be applied to high-stress situations, such as on their morning commute. In the example where an individual reaches a higher stress level on their morning commute, the individual may choose to listen to such songs that they have observed to lower their stress level during their commute in order to lower their stress level in what would be an otherwise stressful time.

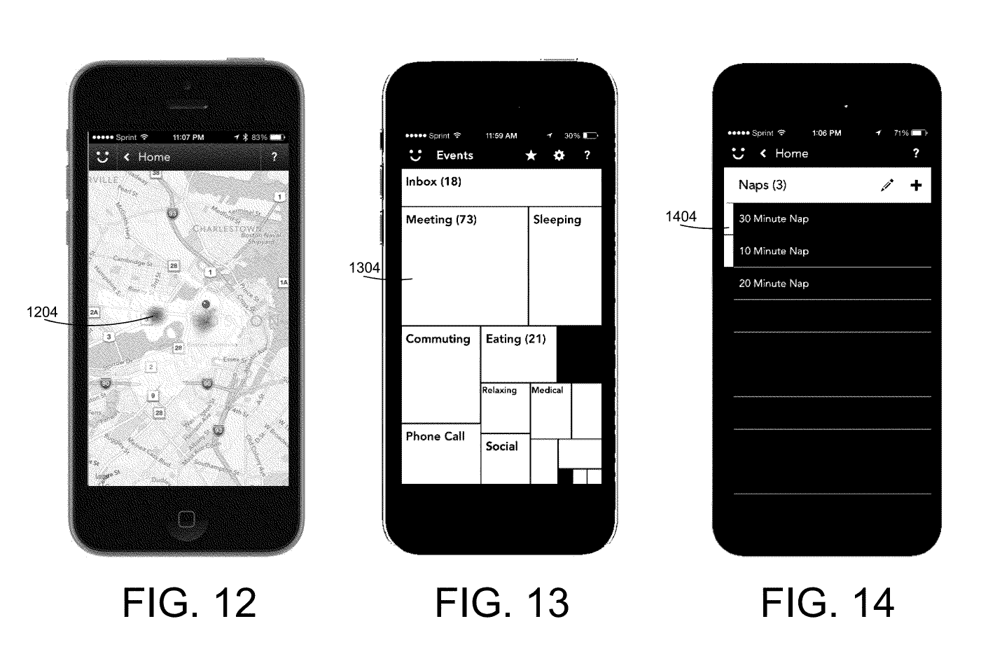

FIG. 12 illustrates yet another example of a user interface presented by an application using a common color scheme to represent health state information. In this example, the application provides a map. The map is marked with points indicating user health state information detected at multiple times. The health state information may be presented as a colored dot in locations where the health state was measured. The pattern of dots may present on the user interface clusters showing locations of particular health states. For example, cluster 1204 may represent a cluster of red dots, indicating a location of high stress. Other clusters may be of other colors, revealing user locations in which the user is under stress or relaxed.

Though the user interface of FIG. 12 may present data for a single user, in other embodiments, the dots representing health state may represent health state measurements taken for multiple users in a population, as described above.

FIG. 13 is yet another example of an application that may use color to represent health state information. In this example, the application determines events engaged in by a user. A computing device may determine when a user is engaged in various bands and track health state of the user at times associated with performing those events. The application may present objects representing various events, with each object colored based on the health state information. For example, object 1304 represents meetings. In this example, multiple objects representing a events over some time interval are presented as a mosaic. For example, the mosaic may represent the way in which a user spends a single day or may represent average time usage over a longer interval such as a week or month. Further, the objects may be sized in proportion to the amount of time spent by the user engaged in the events. Such a mosaic may aid a user in identifying events that impact stress level as an aid to adjusting the user's daily schedule.

FIG. 14 is yet another example of a user interface presented by an application. In this example, the user interface presents tasks that a user may elect to schedule, such as in the calendar application. Each of the tasks may be associated with health state information, which may be represented by an object, such as object 1404. The object may be color-coded to indicate a projected impact on the user's health state. In this example, the tasks from which the user may select are naps of varying length. Each of the options is associated with a color code indicating the impact on the user's stress level of taking a nap of the indicated length. Such information may be obtained from a log of historical data kept for the user, or may be derived from some other source, such as research indicating the impact of naps on individuals.

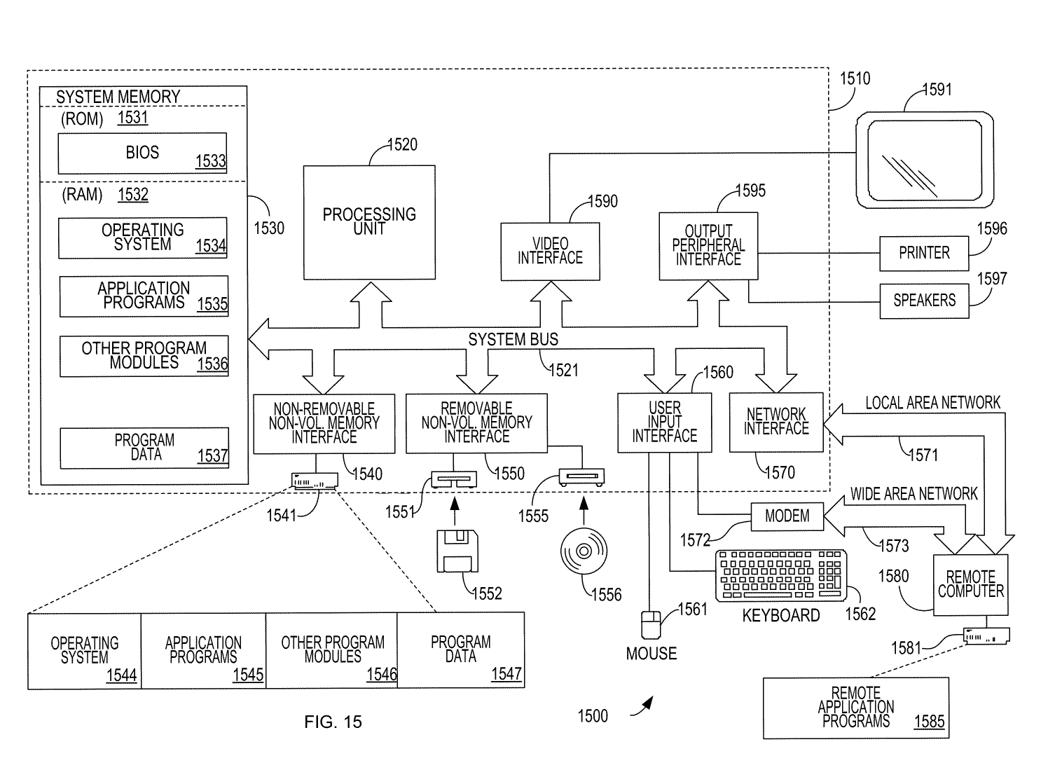

The exemplary user interfaces are illustrated on a portable electronic device, such as the smart phone, processing and display of information as described herein may be controlled by any suitable computing device. FIG. 15 illustrates an example of a suitable computing system environment 1500 on which the invention may be implemented. The computing system environment 1500 is only one example of a suitable computing environment and is not intended to suggest any limitation as to the scope of use or functionality of the invention. Neither should the computing environment 1500 be interpreted as having any dependency or requirement relating to any one or combination of components illustrated in the exemplary operating environment 1500.

The invention is operational with numerous other general purpose or special purpose computing system environments or configurations. Examples of well-known computing systems, environments, and/or configurations that may be suitable for use with the invention include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

With reference to FIG. 15, an exemplary system for implementing the invention includes a general purpose computing device in the form of a computer 1510. Components of computer 1510 may include, but are not limited to, a processing unit 1520, a system memory 1530, and a system bus 1521 that couples various system components including the system memory to the processing unit 1520. The system bus 1521 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 1510 typically includes a variety of computer readable media. Computer readable media can be any available media that can be accessed by computer 1510 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 1510. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 1530 includes computer storage media in the form of volatile and/or nonvolatile memory such as read only memory (ROM) 1531 and random access memory (RAM) 1532. A basic input/output system 1533 (BIOS), containing the basic routines that help to transfer information between elements within computer 1510, such as during start-up, is typically stored in ROM 1531. RAM 1532 typically contains data and/or program modules that are immediately accessible to and/or presently being operated on by processing unit 1520. By way of example, and not limitation, FIG. 15 illustrates operating system 1534, application programs 1535, other program modules 1536, and program data 1537.

The computer 1510 may also include other removable/non-removable, volatile/nonvolatile computer storage media. By way of example only, FIG. 15 illustrates a hard disk drive 1541 that reads from or writes to non-removable, nonvolatile magnetic media, a magnetic disk drive 1551 that reads from or writes to a removable, nonvolatile magnetic disk 1552, and an optical disk drive 1555 that reads from or writes to a removable, nonvolatile optical disk 1556 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 1541 is typically connected to the system bus 1521 through an non-removable memory interface such as interface 1540, and magnetic disk drive 1551 and optical disk drive 1555 are typically connected to the system bus 1521 by a removable memory interface, such as interface 1550.

The drives and their associated computer storage media discussed above and illustrated in FIG. 15, provide storage of computer readable instructions, data structures, program modules and other data for the computer 1510. In FIG. 15, for example, hard disk drive 1541 is illustrated as storing operating system 1544, application programs 1545, other program modules 1546, and program data 1547. Note that these components can either be the same as or different from operating system 1534, application programs 1535, other program modules 1536, and program data 1537. Operating system 1544, application programs 1545, other program modules 1546, and program data 1547 are given different numbers here to illustrate that, at a minimum, they are different copies. A user may enter commands and information into the computer 1510 through input devices such as a keyboard 1562 and pointing device 1561, commonly referred to as a mouse, trackball or touch pad. Other input devices (not shown) may include a microphone, joystick, game pad, satellite dish, scanner, or the like. These and other input devices are often connected to the processing unit 1520 through a user input interface 1560 that is coupled to the system bus, but may be connected by other interface and bus structures, such as a parallel port, game port or a universal serial bus (USB). A monitor 1591 or other type of display device is also connected to the system bus 1521 via an interface, such as a video interface 1590. In addition to the monitor, computers may also include other peripheral output devices such as speakers 1597 and printer 1596, which may be connected through a output peripheral interface 1595.

The computer 1510 may operate in a networked environment using logical connections to one or more remote computers, such as a remote computer 1580. The remote computer 1580 may be a personal computer, a server, a router, a network PC, a peer device or other common network node, and typically includes many or all of the elements described above relative to the computer 1510, although only a memory storage device 1581 has been illustrated in FIG. 15. The logical connections depicted in FIG. 15 include a local area network (LAN) 1571 and a wide area network (WAN) 1573, but may also include other networks. Such networking environments are commonplace in offices, enterprise-wide computer networks, intranets and the Internet.

When used in a LAN networking environment, the computer 1510 is connected to the LAN 1571 through a network interface or adapter 1570. When used in a WAN networking environment, the computer 1510 typically includes a modem 1572 or other means for establishing communications over the WAN 1573, such as the Internet. The modem 1572, which may be internal or external, may be connected to the system bus 1521 via the user input interface 1560, or other appropriate mechanism. In a networked environment, program modules depicted relative to the computer 1510, or portions thereof, may be stored in the remote memory storage device. By way of example, and not limitation, FIG. 15 illustrates remote application programs 1585 as residing on memory device 1581. It will be appreciated that the network connections shown are exemplary and other means of establishing a communications link between the computers may be used.

Having thus described several aspects of at least one embodiment of this invention, it is to be appreciated that various alterations, modifications, and improvements will readily occur to those skilled in the art.

For example, it is described that an emotional state of a user is represented as a color. It should be appreciated that the state may be represented in other ways that allows a common representation, or common interpretation, by different applications. For example, the state may be represented by a value selected from an enumerated set of values. Any application may be configured to recognize, and respond to, each of the members of the enumerated set. A response may include controlling a display color or other display attribute. Alternatively, the state may be represented as a number within a range, such that the location within the range indicates a degree of stress or other emotional and/or physiological characteristic.

As another example, embodiments of a user interface technique were described in which the velocity of a swipe indicates the granularity of time information presented on the display by a calendar application. A similar user interface technique may be applied to control the granularity of other types of information displayed.

Such alterations, modifications, and improvements are intended to be part of this disclosure, and are intended to be within the spirit and scope of the invention. Further, though advantages of the present invention are indicated, it should be appreciated that not every embodiment of the invention will include every described advantage. Some embodiments may not implement any features described as advantageous herein. Accordingly, the foregoing description and drawings are by way of example only.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present invention as discussed above. As used herein, the term “computer-readable storage medium” encompasses only a computer-readable medium that can be considered to be a manufacture (i.e., article of manufacture) or a machine. Alternatively or additionally, the invention may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

What is claimed is:

1. At least one non-transitory computer-readable storage medium storing computer-executable instructions that, when executed, implement a platform for at least one computing device, wherein the platform is configured to perform a method for interacting with a plurality of applications on the at least one computing device, the method comprising:
receiving health information representative of a health state of an individual;
receiving context information from at least one application of the plurality of applications;
generating an indication of the health state based, at least in part, on the received health information and the context information by selecting a value from a set of values representative of different health states; and
providing, through a programming interface, the indication to at least a portion of the plurality of applications.

2. The at least one non-transitory computer-readable medium of claim 1, wherein the method further comprises selecting the value by analyzing previously acquired health state information.

3. The at least one non-transitory computer-readable medium of claim 2, wherein the method further comprises receiving context information corresponding to a time associated with the received health information, and selecting the value comprises selecting a portion of previously acquired health information having context information similar to the received context information.

4. The at least one non-transitory computer-readable medium of claim 3, wherein the context information comprises information indicative of a type of activity associated with the individual at the time.

5. The at least one non-transitory computer-readable medium of claim 2, wherein the portion of previously acquired health information includes health state information of the individual.

6. The at least one non-transitory computer-readable medium of claim 2, wherein the portion of previously acquired health information is health state information from a population having at least one common characteristic identified by context information associated with the previously acquired health information.

7. The at least one non-transitory computer-readable medium of claim 1, wherein the computer platform comprises a portion of a computer operating system.

8. The at least one non-transitory computer-readable medium of claim 1, wherein the set of values is an enumerated set of predetermined values.

9. The at least one non-transitory computer-readable medium of claim 8, wherein the enumerated set of predetermined values corresponds to a set of different colors, wherein each color of the set of colors is associated with a different health state.

10. The at least one non-transitory computer-readable medium of claim 9, wherein each color of the set of colors map to a different stress level.

11. The at least one non-transitory computer-readable medium of claim 1, further comprising computer-executable instructions, that, when executed, implement a plurality of applications, each of the plurality of applications being configured to render a display with a common visual characteristic identified by the indication representative of the health state.

12. A portable electronic system associated with a user, the portable electronic system being configured to execute a plurality of applications, and the portable electronic system comprising:
at least one sensor configured to provide health information representative of a health state of the user; and
at least one processor configured to:
receive context information from at least one application of the plurality of applications;
generate an indication of the health state based, at least in part, on the health information and the context information; and
present a platform interface common to the plurality of applications, wherein the platform interface is configured to enable the plurality of applications to access the indication.

13. The portable electronic system of claim 12, further comprising:
a display configured to present at least one graphical user interface rendered by the plurality of applications; and
a plurality of applications executing on the at least one processor, wherein the applications being configured to render on the at least one graphical user interface visual elements based on the indication.

14. The portable electronic system of claim 13, wherein the at least one graphical user interface includes a plurality of graphical user interfaces associated with the applications and the visual element is a common representation among the plurality of graphical user interfaces.

15. The portable electronic system of claim 12, wherein generating the visual indication of the health state comprises identifying a portion of previously acquired health information based, at least in part, on the context information.

16. The portable electronic system of claim 15, wherein the at least one application is a calendar application and receiving context information provided by the at least one application comprises identifying a type of activity for the user at a time associated with the health information.

17. The portable electronic system of claim 15, wherein receiving context information provided by at least one application of the plurality of applications comprises identifying a location of the user at a time associated with the health information.

18. The portable electronic system of claim 12, wherein generating the visual indicator comprises selecting a value associated with the health information, wherein the value is selected from an enumerated set of values representative of different health states.

19. The portable electronic system of claim 18, wherein the enumerated set of values corresponds to a set of different colors, wherein each color of the set of colors is associated with a different health state.

20. The portable electronic system of claim 12, wherein presenting the at least one graphical user interface comprises mapping the visual indication to a feature within the at least one graphical user interface.

21. The portable electronic system of claim 20, wherein the feature within the at least one graphical user interface includes multiple visual elements positioned to represent time, wherein each of the multiple visual elements have a visual characteristic selected based on the indication at an associated time.

22. The portable electronic system of claim 20, wherein the feature within the at least one graphical user interface is indicative of a type of activity associated with an application the user performs with the application.

23. The portable electronic system of claim 20, wherein the feature within the at least one graphical user interface is indicative of an instruction to the user to perform a type of activity with an application.

24. The portable electronic system of claim 20, wherein the feature within the at least one graphical user interface is indicative of a location associated with the health information of the user while at the location.

25. The portable electronic system of claim 20, wherein the feature within the at least one graphical user interface is indicative of locations associated with health information for a plurality of individuals at the locations.

26. The portable electronic system of claim 12, wherein presenting the at least one graphical user interface comprises presenting a visual indicator corresponding to an average health state for health information acquired over a period of time.

27. The portable electronic system of claim 12, wherein presenting the at least one graphical user interface comprises presenting a plurality of values associated with health information acquired over time in the form of a graph, wherein the values are selected from an enumerated set of values representative of different health states.

28. The portable electronic system of claim 12, wherein presenting the at least one graphical user interface comprises presenting visual indicators for a predicted health state for a future time based, at least in part, on the acquired health information.

29. The portable electronic system of claim 12, wherein the at least one sensor comprises a biosensor configured as a wearable device.

30. The portable electronic system of claim 12, wherein the at least one sensor is coupled to the at least one processor via a wireless link.

31. A server comprising:
at least one processor configured to receive health information indicative of health states of at least one individual over time; and
at least one storage medium storing processor-executable instructions that, when executed by the at least one processor, perform a method comprising:
identifying a portion of the health information based, at least in part, on user health data provided by at least one sensor associated with a user;
generating an indicator for the user health data by analyzing the portion of health information and relating the user health data to the portion of health information; and
transmitting the indicator to at least one portable electronic device over a wide area network, wherein the at least one portable electronic device is associated with the user.

32. The server of claim 31, wherein receiving health information indicative of health states of at least one individual over time comprises receiving health information for a plurality of individuals, identifying the portion of health information comprises identifying health information for a population among the plurality of individuals, and generating the indicator comprises analyzing variation in health information for the population and relating the user health data to the variation.

33. The server of claim 31, wherein receiving health information indicative of health states of at least one individual over time comprises receiving health information for the user, identifying the portion of health information comprises identifying health information for the user, and generating the indicator comprises analyzing variation in health information for the user and relating the user health data to the variation.

34. The server of claim 31, wherein identifying the portion of the health information comprises selecting the portion based on context information associated with the user health data.

35. The server of claim 31, wherein generating the indicator comprises determining a set of values corresponding to a range of health states for the portion of health information and relating the user health data to the range.

36. The server of claim 31, wherein generating the indicator comprises normalizing the user health data to the portion of health information.

37. The server of claim 31, wherein generating the indicator comprises selecting a color from a set of colors, wherein each color of the set of colors is associated with a different health state selected based at least in part on the portion of health information.

38. The at least one non-transitory computer-readable medium of claim 37, wherein each color of the set of colors map to a different stress level.
